(19) United States
(12) Patent Application Publication
(10) Pub. No.: US 2016/0138105 A1
(43) Pub. Date: May 19, 2016

(54) SYSTEM AND METHODS FOR DETERMINING A WOMAN'S RISK OF ANEUPLOID CONCEPTION

(71) Applicants: RAJIV MCCOY, Redwood City, CA (US); DMITRI PETROV, Palo Alto, CA (US)

(72) Inventors: RAJIV MCCOY, Redwood City, CA (US); DMITRI PETROV, Palo Alto, CA (US)

(73) Assignee: The Board of Trustees of the Leland Stanford Junior University, Palo Alto, CA (US)

(21) Appl. No.: 14/938,842

(22) Filed: Nov. 11, 2015

Related U.S. Application Data
(60) Provisional application No. 62/080,251, filed on Nov. 14, 2014.

(51) Int. Cl. C12Q 1/68; G06F 19/24; G06F 19/22

(52) U.S. Cl. CPC C12Q 1/6883; G06F 19/22; G06F 19/24; C12Q 2600/156; C12Q 2600/112

(57) ABSTRACT

The present invention provides methods and systems for determining a woman's risk of carrying an aneuploid embryo based on the maternal genotype, maternal age and optionally paternal age.

### 1. CROSS-REFERENCE TO RELATED APPLICATION

This application claims priority and other benefits from U.S. Provisional Patent Application Ser. No. 62/080,251 filed Nov. 14, 2014, entitled “System and methods for determining a woman's risk of aneuploid conception”. Its entire content is specifically incorporated herein by reference.

### 2. TECHNICAL FIELD OF THE INVENTION

The present invention relates to a system and methods for determining a woman's risk of conceiving an aneuploid embryo and for determining a woman's overall probability to conceive an euploid embryo, as well as fertility-related consequences of these probabilities.

### 3. BACKGROUND

Aneuploidy, the inheritance of an atypical chromosome complement, is a common occurrence in early human embryonic development and is considered the leading cause of pregnancy loss and congenital birth defects. This pattern is driven mostly by errors arising during maternal meiosis, which arrests at the diplotene stage until it resumes at ovulation many years later (Hassold & Hunt, 2001). It has long been established that incidence of aneuploidy affecting maternal chromosome copies increases with maternal age (Penrose et al., 1933), typically beginning after age 30, driven primarily by errors of maternal meiotic origin (Erickson, 1978; Hassold & Chiu, 1985). Approximately 75% of embryos are at least partially aneuploid by day 3, due to prevalent errors of both meiotic and post-zygotic origin (Voullaire et al, 2000; Wells & Delhanty, 2000).

Given the strong implications for successful family planning, a clear understanding of the rates and molecular mechanisms contributing to the various forms of aneuploidy is an important goal in reproductive medicine. In addition to environmental and demographic factors, such as maternal age, recent work demonstrated that genetic factors also influence aneuploidy incidence, such that genotype at these informative loci can be utilized to help predict aneuploidy risk. This information will be helpful in a woman's decision making regarding general family planning and, in particular, whether and when to utilize assisted reproductive technologies such as in vitro fertilization.

### 4. SUMMARY OF THE INVENTION

In one aspect of the invention, a method is provided to determine a woman's susceptibility to conceive an aneuploid embryo based on the presence of a polymorphic allele that is correlated with probability of mitotic error—a common aneuploidy-generating process. In one embodiment of the invention, the polymorphic allele comprises a single nucleotide polymorphism within a regulatory region associated with the PLK4 locus. In a particular embodiment of the invention, the single nucleotide polymorphism is rs2305957 or a genetic variant in linkage disequilibrium with rs2305957.

In a further aspect of the invention, a method is provided to determine a woman's susceptibility to conceive an aneuploid embryo based on the presence of a polymorphic allele at single nucleotide polymorphism rs2305957 or a genetic variant in linkage disequilibrium with rs2305957 as well as accounting for the effect of the woman's age (maternal age) and, optionally, with the prospective father's age (paternal age). In one embodiment of the invention, a statistical model is fit to a large dataset of previous cases to describe the combined effects of genotype at a polymorphic locus, maternal age, and paternal age on the proportion of aneuploid embryos per family undergoing IVF. In a particular embodiment of the invention, the single nucleotide polymorphism is rs2305957 or a genetic variant in linkage disequilibrium with rs2305957. In one embodiment, prediction of aneuploidy risk is achieved for new cases using a statistical model and prediction precision is estimated by resampling the data with replacement multiple times. In other embodiments, probability of inviable aneuploidy, probability of various viable aneuploidies, probability of miscarriage, average time to successful conception with unprotected intercourse timed near ovulation, and other aneuploidy-associated fertility outcomes constitute the predicted variables as all of these are related to aneuploidy status.

In another aspect, a kit is provided for assessing a woman's susceptibility to conceive an aneuploid embryo comprising reagents, probes and instructions for determining the presence or absence of a polymorphic allele in a biological sample from said woman, with the polymorphic allele being located on chromosome 4 and the polymorphic allele's presence indicating the woman's susceptibility to conceive an aneuploid embryo. In a particular embodiment of the invention, the single nucleotide polymorphism is rs2305957 or a genetic variant in linkage disequilibrium with rs2305957.

In a further aspect, a system is provided for determining a woman's susceptibility to conceive an aneuploid embryo comprising: a computing environment, an input device, connected to the computing environment, to receive data from a user, wherein the data received comprises items of information from a woman to provide a profile for said woman, comprising information such as the woman's genotype including the presence or absence of a polymorphic allele, the woman's age and, optionally, prospective father's age, an output device, connected to the computing environment, to provide information to the user; and a computer readable storage medium having stored thereon at least one algorithm to provide for comparing the woman's profile to a library of profiles known to provide an indication of a woman's susceptibility to carry an aneuploid embryo, wherein the system provides results that can be used for determining a woman's susceptibility to conceive an aneuploid embryo.

The above summary is not intended to include all features and aspects of the present invention nor does it imply that the invention must include all features and aspects discussed in this summary.

### 5. INCORPORATION BY REFERENCE

All publications mentioned in this specification are herein incorporated by reference to the same extent as if each individual publication or patent application was specifically and individually indicated to be incorporated by reference.

### 6. DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates two mechanisms that are thought to contribute to aneuploidy: maternal meiotic non-disjunction and mitotic anaphase lag. Maternal meiotic non-disjunction, the failure of homologous chromosomes or sister chromatids to separate, results in maternal chromosome loss in one daughter cell and corresponding maternal chromosome gain in the other daughter cell. Trisomies with a meiotic origin can be identified when both maternal homologs are detected in the same genomic region. Mitotic anaphase lag refers to the delayed movement of a chromatid toward the spindle pole and can result in chromosome loss in one daughter cell. This can occur when microtubules emanating from multiple spindle poles attach to a single kinetochore. Such ‘merotelic’ attachments are more common in the presence of extra centrosomes and other centrosome abnormalities. Because paternal meiotic errors are rare, the absence of paternal chromosome copies can be attributed to mitotic error (but not necessarily anaphase lag) with high confidence.

FIG. 2 shows characteristics of the mitotic-error phenotype, defined as any aneuploidy where a paternal chromosome copy is affected. A. Aneuploidies where paternal chromosomes are affected include an excess of chromosome losses compared to chromosome gains which is consistent with the signature of anaphase lag, described in FIG. 1. Paternal chromosome loss (paternal monosomy) commonly co-occurs with other forms of chromosome loss including maternal monosomy and nullisomy as complex aneuploidies with multiple chromosomes affected. B. Blastomeres with aneuploidies affecting paternal chromosomes (blue; putative mitotic-origin aneuplodies) often contain multiple aneuploid chromosomes in contrast to aneuploid blastomeres in which no paternal chromosome copies are affected (red; predominantly meiotic-origin aneuploidies). Heights of bars indicate densities (i.e., relative frequencies) such that the heights of all bars of a given color sum to one. C. Aneuploidies in which paternal chromosome copies are affected do not increase in frequency with increasing maternal age, while other aneuploidies increase sharply in frequency beginning in the mid-thirties. Error bars indicate standard errors of proportions.

FIG. 3 shows Manhattan and QQ plots depicting P-values of association tests of each genotyped SNP versus the rate of aneuploidy affecting paternal chromosomes, a strong proxy for mitotic aneuploidy. Genes of interest on chromosome 4 (chr4) include INTU, SLC25A31, HSPA4L, LARP1B, PGRMC2 and, particularly, PLK4. P-values are corrected using the genomic control method (Devlin et al., 1999). Results for association with paternal genotype (a control set with approximately the same ethnic composition as the set of female patients) are given in panels A-B, while results for association with maternal genotype are given in panels C-D. For the Manhattan plots (A & C), the red lines represent a standard genome-wide cutoff of 5×10⁻⁸, while the gray dotted lines represent a less stringent P-value of 1×10⁻⁶. The QQ plots (B & D) depict the distributions of P-values observed versus those expected under the null. The gray shaded regions indicate probability bounds. E. Regional association plot for mothers of European ancestry, inferred by comparison to reference populations. Herefore, the association test of the rate of errors affecting paternal chromosome copies was performed by combining genotyped SNPs (square plotting symbols) with imputed SNPs (circular plotting symbols). The purple point indicates the most significant genotyped SNP (rs2305957), and the colors of other variants are based on linkage disequilibrium with this genotyped SNP.

FIG. 4 illustrates effects of genotype on mitotic-error-related phenotypes. To better visualize differences in proportions for boxplots, figures were restricted to include only mothers for whom more than two embryos were tested. A. The proportion of blastomeres per mother with an error affecting a paternal chromosome (a proxy for mitotic aneuploidy) stratified by maternal genotype at the most significant genotyped SNP (rs2305957) for the discovery sample (2,362 individuals and 20,798 embryos with P=8.68×10⁻¹⁶). B. Here is the same phenotype shown as in panel A, replicated in the validation sample (34 individuals and 283 embryos with P=0.0112). C. Panel C shows the mean proportion of blastomeres with an aneuploidy affecting a paternal chromosome versus maternal age, stratified by genotype at rs2305957. Error bars represent standard error of the proportion. D. Panel D shows the mean proportion of aneuploid blastomeres versus maternal age, stratified by genotype at rs2305957. Error bars represent standard error of the proportion. E. Panel E shows the mean number of day-5 trophectoderm biopsies per mother, stratified by genotype at rs2305957 (P=0.00247). Error bars represent standard error.

FIG. 5 illustrates the frequency of alleles at SNP rs2305957 among 1000 Genomes Phase 3 populations. This figure was generated using the Geography of Genetic Variants Browser v0.2.

FIG. 6 illustrates logistic regression coefficient estimates (B) for association of SNP genotype at rs2305957 with aneuploidy affecting any paternal chromosome copy (paternal monosomy, paternal trisomy, or paternal uniparental disomy). Cases were stratified by total number of aneuploid chromosomes (all other blastomeres are considered as controls). This demonstrates that the previously reported association is mostly driven by complex aneuploidies affecting ≧4 chromosomes.

FIG. 7 shows the proportion of aneuploid blastomeres, stratified by maternal age. Beginning at age 35, the proportion of aneuploid blastomeres increases approximately linearly, with a 3.4% increase in the rate of aneuploidy per year. The difference in rates of aneuploidy between the two respective homozygous genotype classes at rs2305957 is therefore equivalent to the average effect of −1.8 years of age during this timespan. Error bars indicate standard errors of the proportions.

FIG. 8. Associations between clinical indications for preimplantation genetic screening (PGS) and rates of meiotic and mitotic error detected with PGS, controlling for maternal age. Only indications with at least one significant association are depicted. Effect size is measured by an odds ratio, where error incidence for a given referral reason is compared to error incidence for all other referral reasons. Error bars indicating 95% confidence intervals. Stars are used to indicate statistical significance in a logistic GLM: * P<0.05, ** P<0.01, *** P<0.001. Translocation carriers had significantly higher rates of meiotic error than patients referred for other reasons. Patients with previous IVF failure had higher rates of mitotic, but not meiotic error, while patients with recurrent pregnancy loss had higher rates of meiotic (BPH) error at day 5.

FIG. 9 shows that certain chromosomes have elevated rates of aneuploidy independent of the developmental stage at which embryos are sampled. A: Per-chromosome rates of aneuploidy affecting day-3 blastomere (n=25,497) and day-5 trophectoderm (TE) biopsies (n=17,219), compared to published per-chromosome rates of aneuploidy among first-trimester miscarriages (n=273). Miscarriage data are reproduced from Lathi et al., (Lathi et al., 2008) and include all reported autosomal trisomies as well as nine observed monosomies of the X chromosome. The y-axis indicates the percentage of chromosomes affected with any form of aneuploidy compared to all samples of that chromosome for which high-confidence calls could be made. Error bars indicate standard errors of the proportions. B-D: Pairwise comparisons of per-chromosome rates of aneuploidy affecting different developmental stages.

FIG. 10 shows chromosome-specific rates of aneuploidy affecting maternal chromosome copies, which are predominantly meiotic in origin, are negatively correlated with chromosome length, while paternal chromosome errors of predominantly mitotic origin show the opposite pattern.} Error bars indicate standard errors of the proportions. A: Proportion of blastomeres affected with maternal trisomy affecting particular chromosomes. B: Proportion of blastomeres affected with maternal monosomy affecting particular chromosomes. C: Proportion of blastomeres affected with paternal trisomy affecting particular chromosomes. D: Proportion of blastomeres affected with paternal monosomy affecting particular chromosomes. E: Per-chromosome proportion of blastomeres affected with maternal trisomy versus chromosome length (r=−0.443, P=0.0343). F: Per-chromosome proportion of blastomeres affected with maternal monosomy versus chromosome length (r=−0.494, $=0.0166$). G: Per-chromosome proportion of blastomeres affected with paternal trisomy versus chromosome length (r=0.701, P=0.000191). H: Per-chromosome proportion of blastomeres affected with paternal monosomy versus chromosome length (r=0.701, P=0.000191).

FIG. 11 shows that a correlation of chromosome-specific rates of maternal and paternal monosomies and trisomies suggest cytogenetic mechanisms underlying their formation. A: Significant correlation in per-chromosome rates of maternal trisomy and maternal monosomy (r=0.849, P=2.99×10-⁰⁷). B: Significant correlation in per-chromosome rates of paternal trisomy and paternal monosomy (r=0.566, P=0.00491). C: Significant correlation in per-chromosome rates of maternal BPH trisomy and maternal monosomy (r=0.897, P=6.98×10-⁰⁹) D: No significant correlation in per-chromosome rates of rare paternal BPH trisomy and paternal monosomy (r=0.0709, P=0.747).

FIG. 12 illustrates that the maternal-age effect on aneuploidy incidence is chromosome specific, with a bias toward smaller chromosomes evidently due to an increased susceptibility to meiotic error. A: Chromosome-specific incidence of aneuploidy for mothers less than and greater than or equal to 35 years of age. Deviations from the x=y line indicate age effects on aneuploidy incidence, with the steep slope in the data reflecting an interaction between the effects of maternal age and chromosome length on BPH aneuploidy (P=1.00×10⁻⁰⁹). Error bars indicate standard errors of the proportions. B: Coefficient estimates (± standard error) of a logistic regression model testing for an association between rate of aneuploidy and maternal age.

FIG. 13 illustrates that meiotic errors tend to affect few chromosomes while mitotic errors tend to affect many chromosomes simultaneously. A: Proportion of errors affecting maternal (as opposed to paternal) chromosome copies versus the total number of aneuploid chromosomes. Errors affecting intermediate numbers of chromosomes are not biased toward maternal or paternal chromosomes, and are therefore likely mitotic in origin. Errors affecting few or many chromosomes are biased toward maternal chromosomes, and are therefore likely to be meiotic in origin. Errors affecting few chromosomes increase with maternal age, consistent with this interpretation. B: The mean number of aneuploid chromosomes increases with maternal age, but only beginning at approximately age 40. This increase is observed whether including or excluding euploid blastomeres from the analysis. Error bars indicate standard errors of the means.

FIG. 14 shows a Venn diagram demonstrating that multiple forms of aneuploidy commonly co-occur within individual blastomeres. While maternal monosomy and maternal trisomy often occur either together or in isolation, the combination of maternal monosomy, paternal monosomy, and nullisomy is a common form of complex aneuploidy affecting 1,437 blastomeres. The co-occurrence of these three forms of chromosome loss, as well as the co-occurrence of maternal chromosome gain and loss are highlighed in black.

FIG. 15 illustrates that complex aneuploidy is more common in blastomere samples than trophectoderm samples. A: Total rate of aneuploidy according to total number of chromosomes affected, stratified by sample type. B: The relative difference between rates of aneuploidy affecting trophectoderm versus blastomere samples. More complex aneuploidies affecting greater numbers of chromosomes are increasingly rare among trophectoderm samples, suggesting inviability and/or self-correction of increasingly complex aneuploidies.

FIG. 16 shows rates of various forms of aneuploidy in blastomere samples with respect to maternal and paternal ages. Error bars indicate standard errors of the proportions. Age groups including fewer than 10 embryos were not plotted to improve figure clarity. A: Errors affecting maternal chromosome copies increased sharply with maternal age. Nullisomies were also significantly more frequent with increasing maternal age. B: Errors affecting maternal chromosome copies also increased with paternal age, as expected given the correlation between maternal and paternal ages. C: Maternal meiotic-origin (BPH) trisomies increased with maternal age. D: Paternal meiotic-origin (BPH) trisomies were extremely rare and showed no significant relationship with paternal age.

FIG. 17 shows evidence of a family-specific effect on aneuploidy risk after controlling for parental ages. Observed across-family variance in aneuploidy rates versus distributions of variance after permuting ploidy status across families, matched and unmatched for parents' ages. Excess variance unexplained by sampling noise and maternal and paternal ages must be attributable to uncharacterized environmental and/or genetic factors influencing aneuploidy risk.

FIG. 18 and FIG. 19 illustrate various embodiments of the invention to predict the probability of aneuploidy per blastomere in relation to maternal age. These predictions can then be translated to derive the probability of a woman to have an inviable aneuploidy, viable aneuploidy (e.g., Down Syndrome, Edwards Syndrome), her probability of IVF success, the number of embryos required for a high and/or prespecified probability of IVF success, probability of miscarriage, average time to successful conception with unprotected intercourse timed near ovulation, and other aneuploidy-associated fertility outcomes.

### 7. DETAILED DESCRIPTION

The present invention provides methods and systems for determining a woman's genetic susceptibility to conceive an aneuploid embryo.

Genetic polymorphism is known to be associated with an individual's susceptibility to deviations from the norm. Polymorphisms of interest include SNPs, splice variants, deletions, insertions and similar variations.

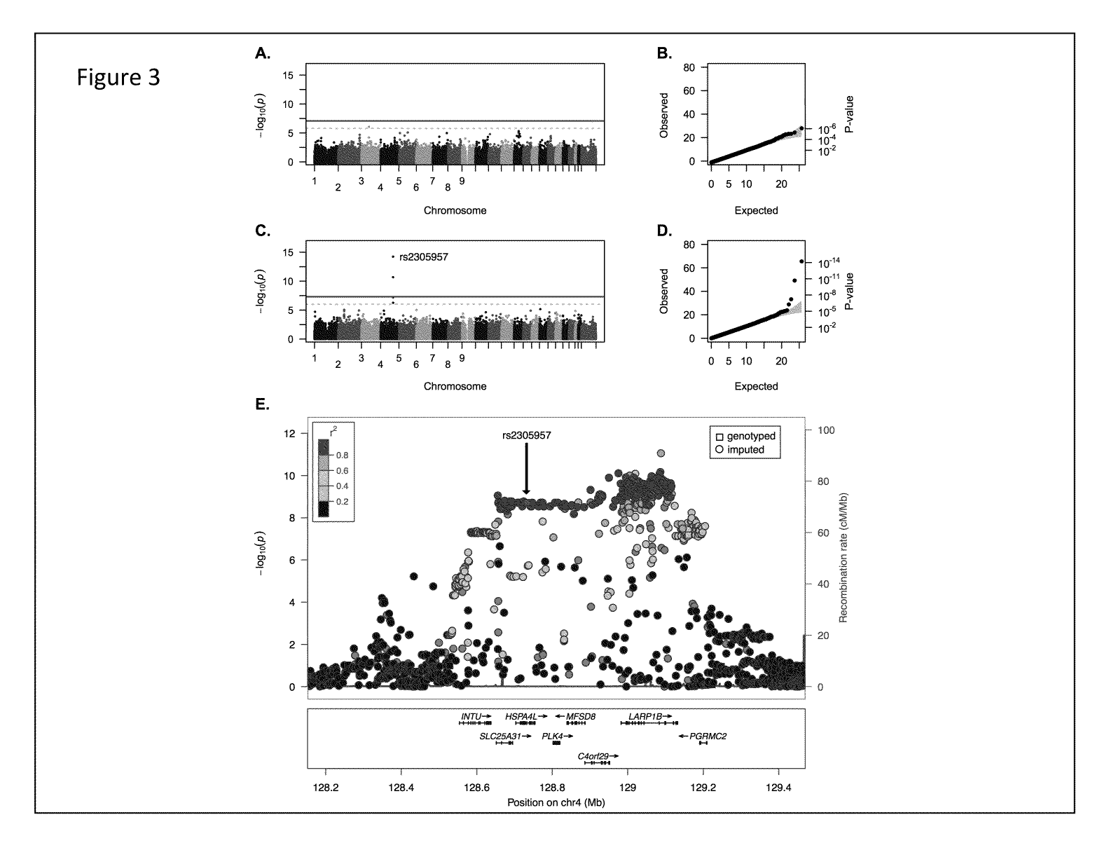

As shown herein, polymorphisms on chromosome 4, such as for example single nucleotide polymorphism rs2305957, are predictive of a woman's susceptibility to conceive an aneuploid embryo, in particular when the maternal age and, optionally, the prospective father's age, are taken into account. The genes PLK4, INTU, SLC25A31, HSPA4L, LARP1B, and PGRMC2 are potential causative candidates (see also FIG. 3E), but the identification of the causative gene is not important for predicting aneuploidy risk as the statistical association is the only relevant factor.

This information will be helpful in a woman's decision making regarding general family planning and, in particular, whether and when to utilize assisted reproductive technologies such as in-vitro fertilization.

Before describing detailed embodiments of the invention, it will be useful to set forth definitions that are utilized in describing the present invention.

### 7.1. Definitions

Unless defined otherwise, all technical and scientific terms used herein have the same meaning as commonly understood by a person of ordinary skill in the art to which this invention BELONGS. The following definitions are intended to also include their various grammatical forms, where applicable. As used herein, the singular forms “a” and “the” include plural referents, unless the context clearly dictates otherwise.

The PLK4 gene encodes a member of the polo family of serine/threonine protein kinases. As a key regulator of centriole biogenesis, the expressed PLK4 protein is required for progression through mitosis, cell survival, and embryonic development.

The INTU gene, inturned planar cell polarity protein, is involved in nervous system development, regulation of keratinocyte proliferation and differentiation, regulation of cell division, limb development and the like.

SLC25A31 is a member of the solute carrier family and, as mitochrondrial ADP/ATP carrier, catalyzes the exchange of cytoplasmic ADP with mitochondrial ATP across the mitochrondrial inner membrane.

HSPA4L is a member of the heat-shock protein 4 like family and is involved in embryonic lung maturation.

ARP1B, actin-related proteinl homolog B, encodes a subunit of dynactin and is involved in chromosome movement and spindle formation.

PGRMC2, progesterone receptor membrane component 2, is implicated in tumor suppression, migration inhibition and regulation of cytochrome P450 enzyme activity.

Polymorphism, as used herein refers to variants, i.e. DNA sequence differences, in the gene sequence. Such variants may include single nucleotide polymorphisms, splice variants, insertions, deletions and transpositions. Polymorphism is generally found between ethnic groups or geographically diverse groups. While having a different sequence, polymorphisms produce gene products that may or may not be functionally equivalent. Particular sequence variants that produce gene products with an altered function are called alleles. A single nucleotide polymorphism (SNP) refers to a polymorphic site that is a single nucleotide in length.

Detection of polymorphism can be achieved by standard techniques of hybridization, sequence analysis, quantitative polymerase chain reaction and the like.

Linkage disequilibrium, as used herein, describes the correlation of genotypes at separate polymorphic genetic loci, such that genotype at one locus may serve as a proxy for genotype at the other polymorphic locus. Physical linkage (i.e. nearby location on a chromosome) is one common, but not exclusive reason that linkage disequilibrium may arise.

A biological sample, as used herein, can be obtained from any source which contains genomic DNA including, not limited to, a blood sample, sample of cerebrospinal fluid, or tissue sample from skin, muscle, buccal, conjunctival mucosa and the like.

### 7.2. Aneuploidy

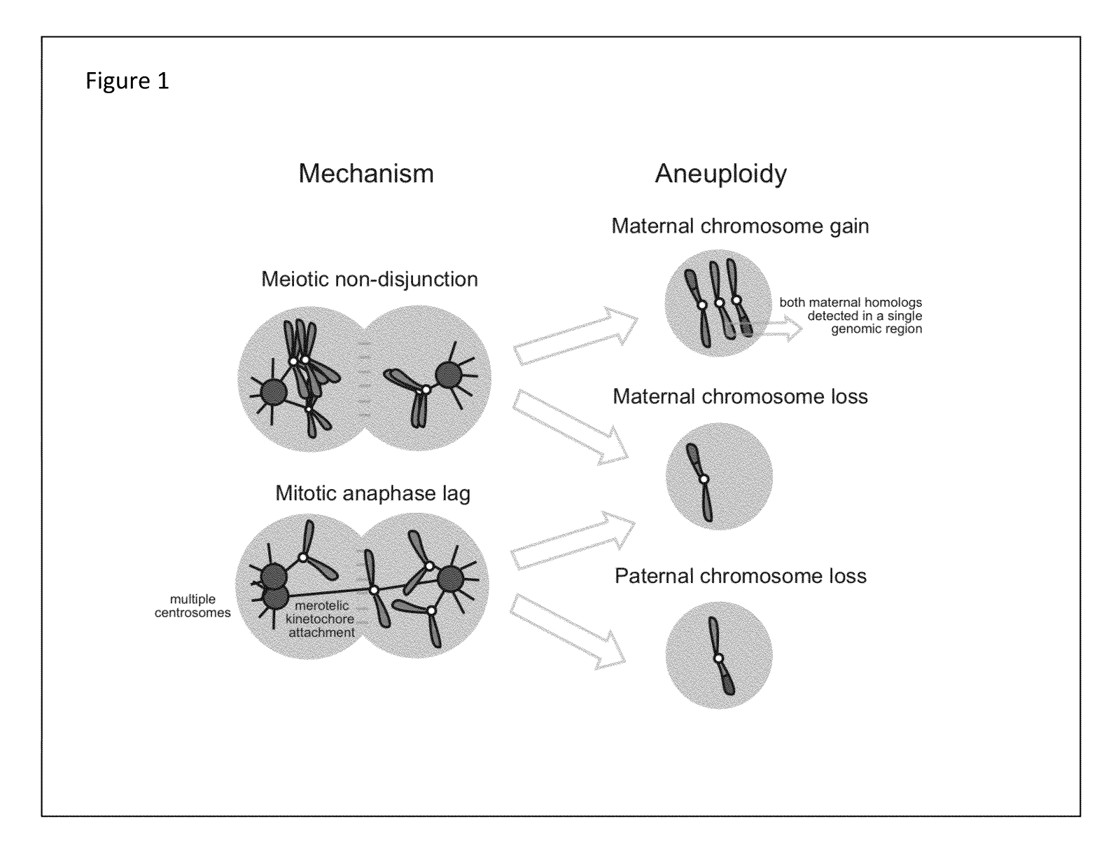

Aneuploidy is an inheritance of an atypical chromosome complement and considered the primary cause of pregnancy loss and decline of fertility with advancing maternal age. As illustrated in FIG. 1, two commonly occurring mechanisms are thought to be the predominant factors leading to aneuploidy: maternal meiotic non-disjunction and mitotic anaphase lag. These mechanisms are considered common based on previous surveys of early embryos (Delhanty et al, 1997; Cupisti et al., 2003; Coonen et al., 2004; Daphnis et al., 2005) as well as studies by the inventors of the present invention. Maternal meiotic non-disjunction, the failure of homologous chromosomes or sister chromatids to separate, results in maternal chromosome loss in one daughter cell and corresponding maternal chromosome gain in the other daughter cell. Trisomies with a meiotic origin can be identified when both maternal homologs are detected in the same genomic region.

Mitotic anaphase lag refers to the delayed movement of a chromatid toward the spindle pole and can result in chromosome loss in one daughter cell. This can occur in cases of erroneous kinetochore attachment, when microtubules emanating from opposite spindle poles attach to a single kinetochore instead of sister kinetochores, creating merotelically attached kinetochores. Merotelic kinetochore orientation that persists until mitotic anaphase impairs separation of chromatids to the opposite spindle poles.

Because paternal meiotic errors are rare, the absence of paternal chromosome copies can be attributed to mitotic error (but not necessarily anaphase lag) with high confidence.

### 7.3 Identification of Associations Between Maternal and Paternal Genotypes and Rates of Aneuploidy

As detailed in Example 1, a genome-wide association study was performed to identify links between parents' genotypes and rates of aneuploidy among day-3 embryos screened during in vitro fertilization (IVF) cycles. Linked variants on chromosome 4, regions q28.1-q28.2 of maternal genomes, were identified that were associated with an elevated rate of complex aneuploidy of putative mitotic origin. Mothers with the high-risk genotypes contributed fewer embryos for testing at day 5 (P=0.00247), suggesting that embryos from those mothers are less likely to survive to blastulation. The association signal spans eight genes, including Polo-like kinase 4 (PLK4), a strong causal candidate given its well-characterized role in the centriole duplication cycle (Habedanck, 2005; Bettencourt, 2005) and its ability to alter mitotic fidelity upon minor misregulation (Firat, 2014). Our study represents the first documented association between natural genetic variation and human aneuploidy risk. Given the known connection between mosaic aneuploidy and pregnancy loss (Santos, 2010), this finding may help explain variation in female fertility.

### 7.4 Prediction of a Woman's Risk to Conceive an Aneuploid Embryo Based on a Comparison to a Library of Profiles (Set of Predictor Variables) Known to Provide an Indication of a Woman's Risk to Carry an Aneuploid Embryo

The invention describes a procedure that can be used to combine information about maternal age and genotype to make diagnostic predictions about aneuploidy risk and fertility. Because rate of aneuploidy is a complex quantitative trait, risk prediction is by nature probabilistic and non-trivial. To achieve this goal, a model is trained using the data from a large reference panel of in-vitro fertilization (IVF) patients and is designed in a such a way that it can be further refined using additional information, such additional genetic and environmental data and information about the success of the procedure itself. For the reference panel, maternal age and genotype at SNP rs2305957 (and linked SNPs in the region around the gene PLK4) are used as predictors in a statistical model, e.g. a linear regression model, where the response variable is the ploidy status of blastomeres. The example model assumes a binomial error distribution with a logit link. A model was built using data from 1095 unrelated patients: logit(Y)=b₀+b₁X₁+b₂X₁²+b₃X₁³+b₄X₂+ where Y is a two-column matrix containing the counts of euploid and aneuploid blastomeres for each mother, X₁ is the maternal age, and X₂ is the maternal genotype, encoded as the number of alternative alleles at SNP rs2305957 (and linked SNPs in the region of PLK4). Both age and genotype are significant predictors of rate of aneuploidy, together explaining 27% of the variance in proportion of aneuploidy per mother (McFadden's pseudo-R²=0.269).

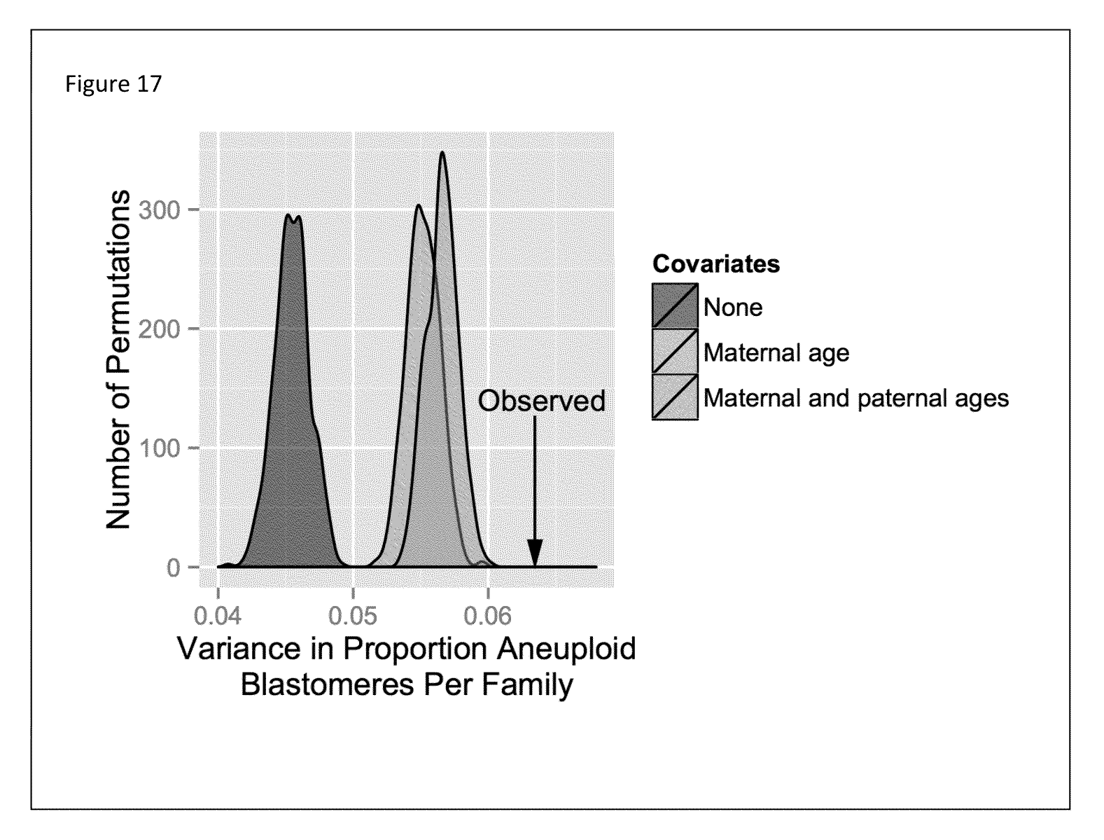

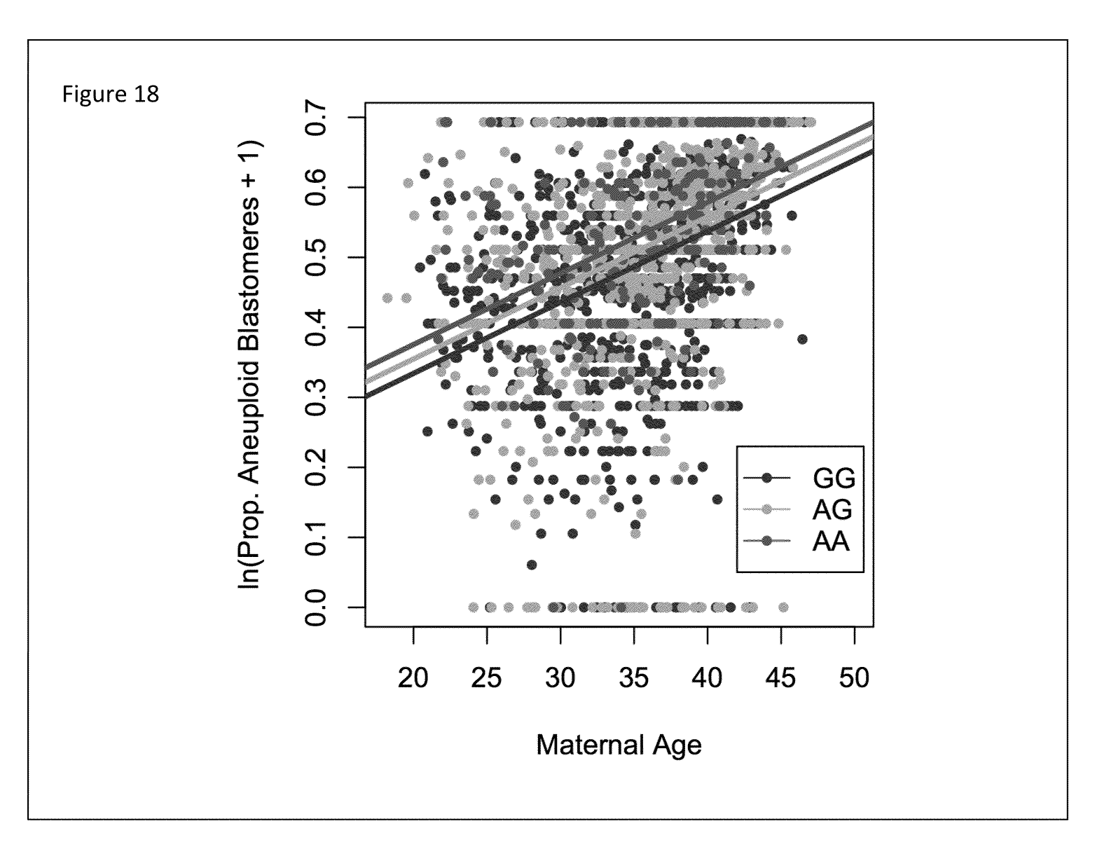

For new cases, regression prediction methods (such as those implemented using the predict.glm function in R) can be used to estimate the probability of aneuploid conception, along with standard error in the estimate based on predictor variables (see FIGS. 17 and 18). To verify the predictive power of our model, we used a set of an additional 1095 unrelated patients whose embryos were screened for aneuploidy using the same procedure. The Pearson correlation between predicted and observed proportions of aneuploid blastomeres per case was highly significant (r=0.436, P<1×10⁻¹⁰, and even stronger when weighting the correlation by sample size (r=0.516). If the mitotic-error associated genotypes are also associated with increased risk of pregnancy loss and other fertility-related phenotypes, the predicted probability of mitotic-error per blastomere can be translated into predictions of the probability of inviable aneuploidy, probability of various viable aneuploidies (e.g. Down syndrome, Edwards syndrome), probability of IVF success, the number of embryos required for a high and/or prespecified probability of IVF success, probability of miscarriage, average time to successful conception with unprotected intercourse timed near ovulation, and other aneuploidy-associated fertility outcomes (see FIGS. 17 and 18). These predictions would be achieved using a procedure analogous to the procedure above, but with the response variable being each of these alternative phenotypes.

An important aspect of the prediction procedure is the estimation of precision in predictions of various outcome variables for new cases. A 95% prediction interval, for example, gives the range within which we can be 95% confident that the response variable will fall given predictors for a new case (including age and informative genotypes). To avoid assumptions about the distribution of the response variable, our procedure uses bootstrap resampling to estimate the prediction interval. This is achieved by repeating the prediction procedure multiple times (e.g. 1,000 bootstrap replicates) on data resampled with replacement from the original dataset and with the same size as the original dataset. Quantiles of the resulting distribution of predicted values then constitute the boundaries of the prediction interval (0.025 and 0.975 quantiles, in the case of a 95% prediction interval). Thus, predictions can be stated along with a measure of precision. This procedure may be used, for example, to determine how certain a couple can be to achieve a euploid live birth from IVF if transferring X number of embryos, with Y maternal age, and Z genotype at the informative SNPs. This can also be restated as the number of cycles and number of embryos that need to be transferred in order to be 95% confident of a euploid live birth. Analogous predictions can be achieved for all of the aforementioned outcome variables probability of inviable aneuploidy, probability of various viable aneuploidies, probability of miscarriage, average time to successful conception with unprotected intercourse timed near ovulation, and other aneuploidy-associated fertility outcomes) at any level of confidence.

In another embodiment, the model can be further refined using publicly-available data as well as fertility history and pregnancy outcome follow-up data from the clients of this service. It needs to be emphasized, however, that individuals using the service do not need to be existing IVF patients. While the precise form of the statistical model (e.g. model coefficients, additional predictor variables, additional outcome variables) may change, the key feature of our model is the combined use of information about parental ages and PLK4 genotype (or other genotypes correlated with PLK4 genotype) to predict aneuploidy risk. The genetic architecture of many quantitative traits is such that many common genetic variants of small effect combine with the one another and the environment (and potentially interact) to produce the phenotype. In many cases, the effects of individual variants are so small that they will not fall below genome-wide significance cutoffs. Methods have therefore been developed to predict phenotypes from all SNPs together by first constructing a pairwise relatedness matrix (based on randomly selected set of unlinked SNPs), then training a model based on that matrix rather than the effects of individually-significant SNPs.

An approach is proposed whereby the training sets for this method are also stratified by maternal age and by the identified SNPs at the PLK4 locus, to appropriately account for the effect of increasing age on aneuploidy. Aneuploidy risk can then be predicted for a new individual using standard regression prediction methods. The aneuploidy phenotypes can also be stratified into mitotic- and meiotic-origin aneuploidy, which likely have distinct genetic determinants. Predicted risks of different forms of aneuploidy can then be translated into predictions of the overall probability of inviable aneuploidy, probability of various viable aneuploidies (e.g. Down syndrome, Edwards syndrome), probability of IVF success, the number of embryos required for a high and/or prespecified probability of IVF success, probability of miscarriage, and average time to successful conception with unprotected intercourse timed to occur around ovulation, if it is verified that these traits are indeed associated with aneuploidy among blastomere samples, as we hypothesize. Again, new cases can be added to the dataset to further refine the model for future use.

### 7.5 Computer-Based System for Determining a Woman's Susceptibility to Conceive an Aneuploid Embryo

A computer-based system for determining a woman's susceptibility to conceive an aneuploid embryo is comprised of a computing environment to which an input device is connected for receiving data from a user comprising information such as the woman's genotypical information, the woman's age and, optionally, the prospective father's age. The computer-based system comprises furthermore an output device such as a computer screen for presenting or visualizing information to the user, further a computer readable storage medium with at least one algorithm to facilitate the comparison of a woman's susceptibility profile to a library of profiles known to provide an indication of a woman's susceptibility to conceive an aneuploid embryo.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### 8. EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### 8.1. Example 1

### Screening for Aneuploidy

### 8.1.1. Experimental Procedures

Described below are the experimental procedures utilized in the determination of a woman's risk of conceiving an aneuploid fetus, based on information of the woman's genetic makeup and age.

In preparation for aneuploidy screening, cells were biopsied from each embryo at either day 3 (individual blastomeres) or day 5 (multi-cell trophectoderm tissue) following conception, followed by whole genome amplification and single nucleotide polymorphism (SNP) genotyping on the HumanCytoSNP-12 BeadChip (Illumina; see under ‘Experimental Procedures’). Genetic material from biological mothers and biological fathers was also collected using buccal swabs or peripheral venipuncture and genotyped on the same SNP-microarray. Combining these data, the Parental Support algorithm (Johnson et al., 2010) was then applied to determine chromosome-level ploidy status of each blastomere. This approach was extensively validated by Johnson et al. (Johnson et al., 2010), who demonstrated that both false-positive and false-negative rates were not statistically different than the ‘gold standard’ method of metaphase karyotyping.

Furthermore, our data were filtered based on confidence scores (see Methods) which were previously demonstrated to strongly correlate with false-detection rates (Johnson, 2010). However, all previous validation was performed for individual blastomeres, so it is unknown how accuracy would be affected in the face of chromosomal mosaicism that could potentially affect multi-cell trophectoderm biopsies. We therefore performed our association study on 2,362 unrelated mothers (1,956 IVF patients and 406 oocyte donors) and 2,360 unrelated fathers meeting genotype quality-control thresholds (see Methods) and from whom at least one day-3 biopsy was obtained with the blastomere prividing a high-confidence result (a total of 20,798 blastomeres).

Sampling and Genotyping of Embryonic Cells Obtained from Various In-Vitro Fertilization Clinics.

After fertilization, single cells were biopsied from separate embryos on day 3, according to the standard protocols of each IVF clinic. Samples were then shipped overnight to Natera, Inc., for prenatal genetic screening (PGS). To minimize contamination, blastomeres were sequentially washed in three drops of hypotonic buffer (5.6 mg/ml KCl, 6 mg/ml bovine serum albumin). DNA was extracted with PKB (Arcturus PicoPure Lysis Buffer, 50 mM DTT) at 56° C. for 1 hour and 95° C. for 10 minutes before gene amplification using a modified Multiple Displacement Amplification (MDA) kit (GE Healthcare) at 30° C. for 2.5 hours, then 65° C. for 15 minutes. Parental DNA samples, obtained from blood draws or MasterAmp buccal tissue swabs (Epicentre), were extracted using a DNeasy Blood and Tissue kit (Qiagen). Parental and embryonic sample DNA was then genotyped on the Illumina HumanCytoSNP-12 BeadChip. For parental samples, genotyping calls were performed using the standard Infinium II protocol from Illumina, Inc., using BeadStudio software.

Screening for Aneuploidy.

The Parental Support algorithm used for copy number determination was previously described by Johnson and coworkers (Johnson et al., 2010). Using this methodology, noisy genotype data from blastomeres is overcome by focusing on informative single nucleotide polymorphism, SNPs, based on parent genotypes, and combining data over large chromosomal windows. This approach also generated confidence scores which were shown to correlate with rates of false-detection (Johnson et al., 2010). To improve detection accuracy, all chromosome calls were masked with confidence scores <80%; furthermore, all blastomeres were removed, if they contained 5 or more low-confidence calls (779 blastomeres did not meet this quality standard). 1,734 detected cases of whole-genome nullisomy were removed as indistinguishable from artefacts of failed amplification.

Discovery Phase.

In preparation for association testing, KING Version 1.4 (Manichaikul et al., 2010) was used to select a random set of unrelated individuals (no individuals of first or second degree relatedness) thereby removing duplicate samples which were otherwise common due to patients undergoing multiple cycles of in-vitro fertilization. PLINK Version 1.90b1g (Purcell et al., 2007) was used to perform a sex check, remove all SNPs with less than 95% call rate, and then to remove samples with less than 95% genotyping efficiency in accordance with genome-wide association studies (GWAS) quality control standards (Turner et al., 2011). As additional quality control and to reduce the multiple-testing burden, SNPs with a frequency of ≦1% were removed. Final quality-filtered sample sizes for mothers and fathers used for subsequent association testing were therefore slightly different.

Sets of blastomeres with different forms of aneuploidy were first defined assuming that errors arising during maternal meiosis would have different underlying genetic architecture than those of post-zygotic mitotic origin. No attempt was made to assign all cases of aneuploidy to these alternative error classes, but instead selected subsets of aneuploid blastomeres that could be assigned to one or the other with high confidence. In the case of maternal meiotic error, all maternal trisomies were considered, where homologs from both maternal grandparents were observed at any chromosomal position in the blastomere. Such aneuploidies should be unambiguously meiotic in origin. Post-zygotic errors in mitosis were assumed to affect maternal and paternal chromosomes approximately equally. Since previous studies demonstrated that errors in male meiosis are rare (≦abnormal sperm, see Templado et al., 2011), a set of blastomeres with putative mitotic error was conservatively identified as one with any aneuploidy affecting the paternal copy of any chromosome. This set of aneuploidies included paternal monosomy, paternal trisomy, and paternal uniparental disomy, even when these errors co-occur with other forms of aneuploidy. No association was observed between the incidence of this set of aneuploidies and maternal age, which lends support to the accuracy of the classification scheme, as employed.

For both the maternal meiotic and post-zygotic error classes, a response variable was defined by assigning all blastomeres in the aneuploid set as cases and all other blastomeres as controls for each IVF cycle. This classification was repeated for all 2,362 unrelated mothers in the data set. The case-control data were then fit in MATLAB Version 7.12.0 (MathWorks, Inc.) using a generalized linear model assuming a binomial error distribution with a logit link function. Upon observing evidence of over-dispersion, the model was refit without fixing the dispersion parameter at 1, i.e., quasi-binomial.

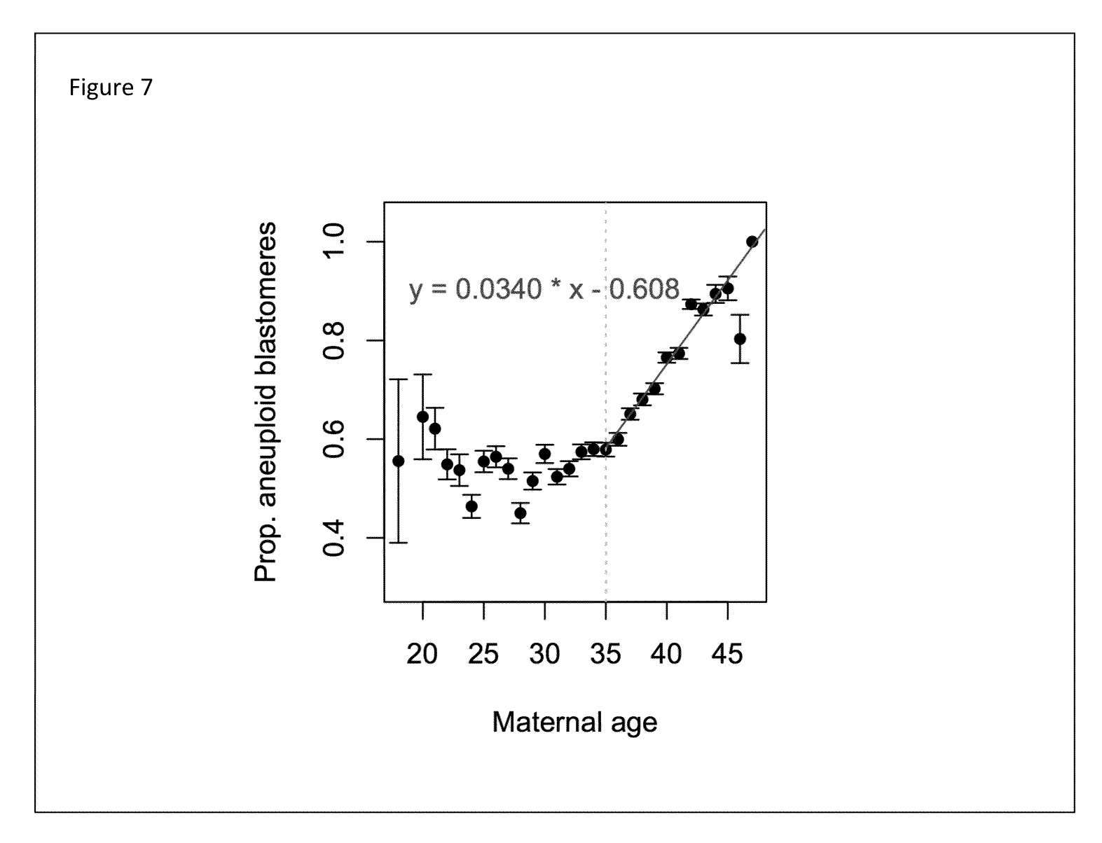

In order to estimate the effect size of genotype on overall aneuploidy in units of maternal age, a line was fit to the plot of proportion of aneuploid blastomeres versus maternal age, for maternal age ≧35 years (FIG. 7), weighting the regression by the square root of the total samples per age category to account for measurement error. The resulting slope was then compared to the difference in the proportion of aneuploid embryos between the two homozygous maternal genotype classes at SNP rs2305957.

Robustness and Statistical Validation.

To exclude population stratification as a possible source of spurious association and to potentially identify population-specific associations, principal components analysis was used to infer ancestry of patients in the sample. First the set of overlapping SNPs between the 11 HapMap population samples and the sample genotypes were extracted. Genotypes were re-encoded as 0, 1, or 2 to reflect the number of alternative alleles carried by each individual at each SNP. The data were randomly downsampled to 20,000 SNPs, then performed principal component analysis (PCA) on the HapMap populations to define the principal component axes. For each individual in the sample, principal component scores were then calculated on these predefined axes. Individuals who fell within the ranges of European or East Asian reference samples were grouped by performing the previous association test on these subsamples of 1,332 and 259 individuals, respectively. Any residual population stratification (quantified by the parameter lambda) was then corrected using the genomic control approach (Devlin et al., 1999).

For the validation step which was performed using data as of March 2014, all new cases since the initial database pull in September 2013 were selected, compiling both genotype data and generating embryonic aneuploidy data by running the aneuploidy classifier algorithm. The genotype data of this new set were then combined with the genotype data of the unrelated individuals used in the discovery stage, using again KING (Manichaikul et al., 2010) to extract a new set of unrelated individuals. The new cases (34 individuals, 283 embryos) were then selected from the resulting set to ensure that duplicate or related individuals were not present across or within the discovery and validation samples.

A generalized linear model was used to test for differences in the number of embryos contributed by mothers with different genotypes at the associated locus. Genotype was encoded as the number of alternative alleles at the SNP rs2305957, thereby testing for an additive effect. As maternal age is also associated with the number of tested trophectoderm biopsies, this effect was controlled by including a second-order polynomial effect of maternal age, such that the model had the form: Y=β₀+B₁(Age)+β₂(Age)²+β₃(Alt. allele count).

The model assumed a Poisson error distribution, modelling overdispersion by not fixing the dispersion parameter (i.e., quasi-Poisson). To test for potential epistatic effects, the model was refit while including genotype at rs2305957 (again encoded as the number of alternative alleles at this locus) as a linear covariate.

As initial genotyping was performed using the ˜300K SNP Illumina Cyto-12 chip, genotypes were relatively sparsely distributed throughout the genome. The association signal was therefore refined by performing genotype imputation. First the 1,332 unrelated individuals falling within the range of the first three principal components of HapMap samples from populations of European ancestry were selected. Using BEAGLE Version 4.r1230 (Howie et al., 2009) untyped markers were imputed based on a European reference panel from the 1,000 Genomes Project (1000 Genomes Project Consortium, 2010). The association tests were then repeated by using both genotyped and imputed sample genotypes, thereby allowing to define the extent of the associated haplotype.

The program SNAP (Johnson et al., 2008) was utilized to identify variants in strong linkage disequilibrium with the most significant genotyped SNP (rs2305957); functional annotations were then retrieved for this list of SNPs using SNPnexus (Chelala et al., 2009). SNP effect predictions were performed using SIFT (Kumar et al., 2009) and PolyPhen2 (Adzhubei et al., 2010), but it is noted that these approaches have known biases against SNPs for which the reference genome carries the derived allele (Simons et al., 2014).

### 8.1.2 Genome-Wide Association Studies

Described below are the results of genome-wide association studies taken into account for the determination of a woman's risk of conceiving an aneuploid fetus, based on information of the woman's genetic makeup and age.

A total of 240,990 SNPs passed quality-control filtering and were used for genome-wide association tests of aneuploidy risk, first to test for associations between the rates of errors of putative maternal meiotic origin and maternal genotypes.

Cases were defined as the set of blastomeres with maternal trisomies where homologs from both maternal grandparents were observed in a single genomic region (FIG. 1). Because errors of meiotic origin increase dramatically with maternal age, age was included as a covariate in the association test. No association achieving genome-wide significance (P-value threshold 5×10⁻⁸) with respect to this meiotic-error phenotype was observed. As a negative control, associations of the same phenotype with paternal genotype were also tested for, and again no results achieving genome-wide significance were observed.

Next associations were tested for between the rates of errors of putative mitotic origin and maternal and paternal genotypes. In this case, genotypes from both parents were of potential interest. The first mitotic divisions of the developing embryo took place under the control of maternal gene products provided to the oocyte, as zygotic genome activation primarily occurs at the 4-8 cell stage (Tadros, 2009). These initial cell divisions are highly error-prone, and it was therefore hypothesized that variation in maternal gene products could contribute to variation in rates of post-zygotic error among embryos from different mothers. However, it is conceivable that paternal genotype could also affect aneuploidy risk, as the centrosome, the microtubule organizing center that controls cell division, is inherited via the sperm (Simerly, 1995). With this in mind, a set of blastomeres was defined with putative mitotic errors similar to those containing any aneuploidy affecting a paternal chromosome copy, excluding paternal trisomies of putative meiotic origin (FIG. 1). Because aneuploidy was estimated to affect fewer than 5% of sperm (Templado, 2011) and because paternal meiotic trisomies were detected for fewer than 1% of blastomeres, this set of aneuploid cases was expected to be nearly exclusively mitotic in origin.

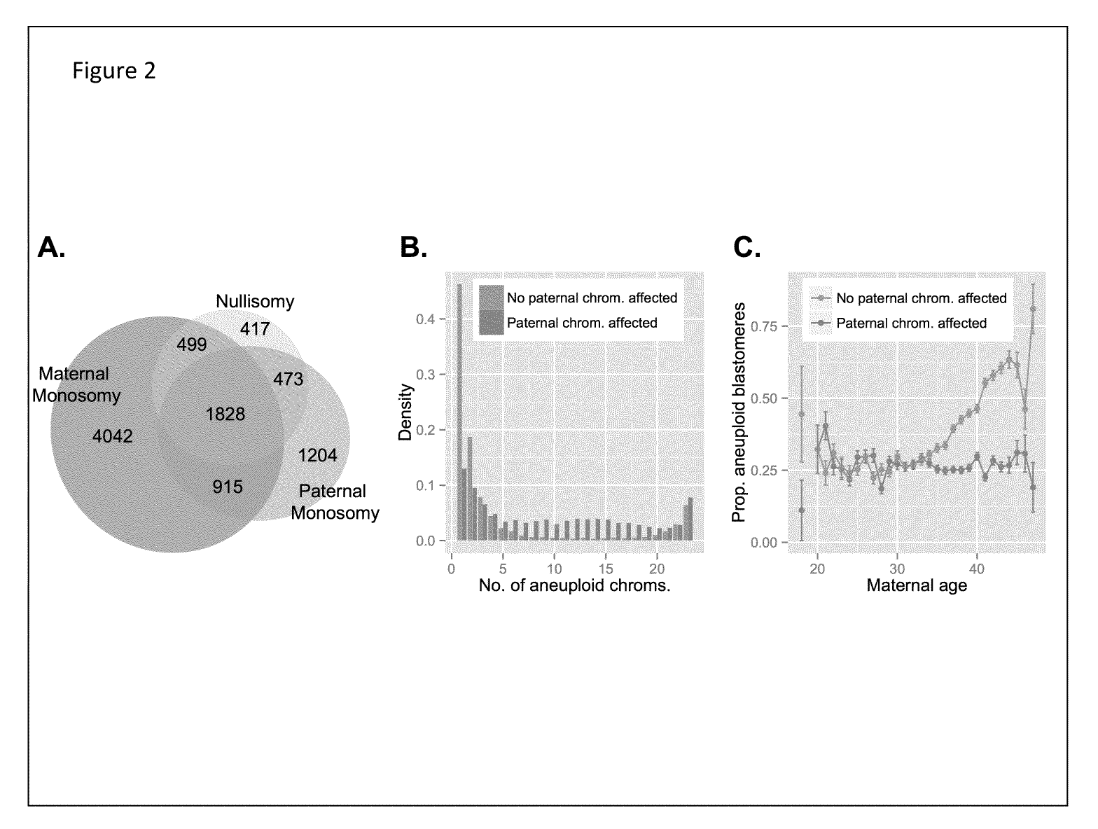

The 5,438 putative mitotic-origin aneuploidies were predominantly characterized by a distinct error profile involving multiple chromosome losses (FIGS. 2A & 2B). A total of 4,420 blastomeres contained at least one paternal monosomy, while only 1,750 of the blastomeres contained at least one paternal trisomy. Of the 4,420 blastomeres with a paternal monosomy, 2,743 (62.1%) also contained at least one maternal monosomy, while 2,301 (52.1%) contained at least one nullisomy (FIG. 2A). All three forms of chromosome loss co-occurred in 1,828 blastomeres (FIG. 2A). While other aneuploidies increase sharply in frequency with increasing maternal age, this subset of mitotic-origin aneuploidies was constant with respect to age (FIG. 2C).

### 8.1.3. Results

Described below is the use of the methods described herein for the determination of a woman's risk of conceiving an aneuploid fetus, based on information of the woman's genetic makeup and age.

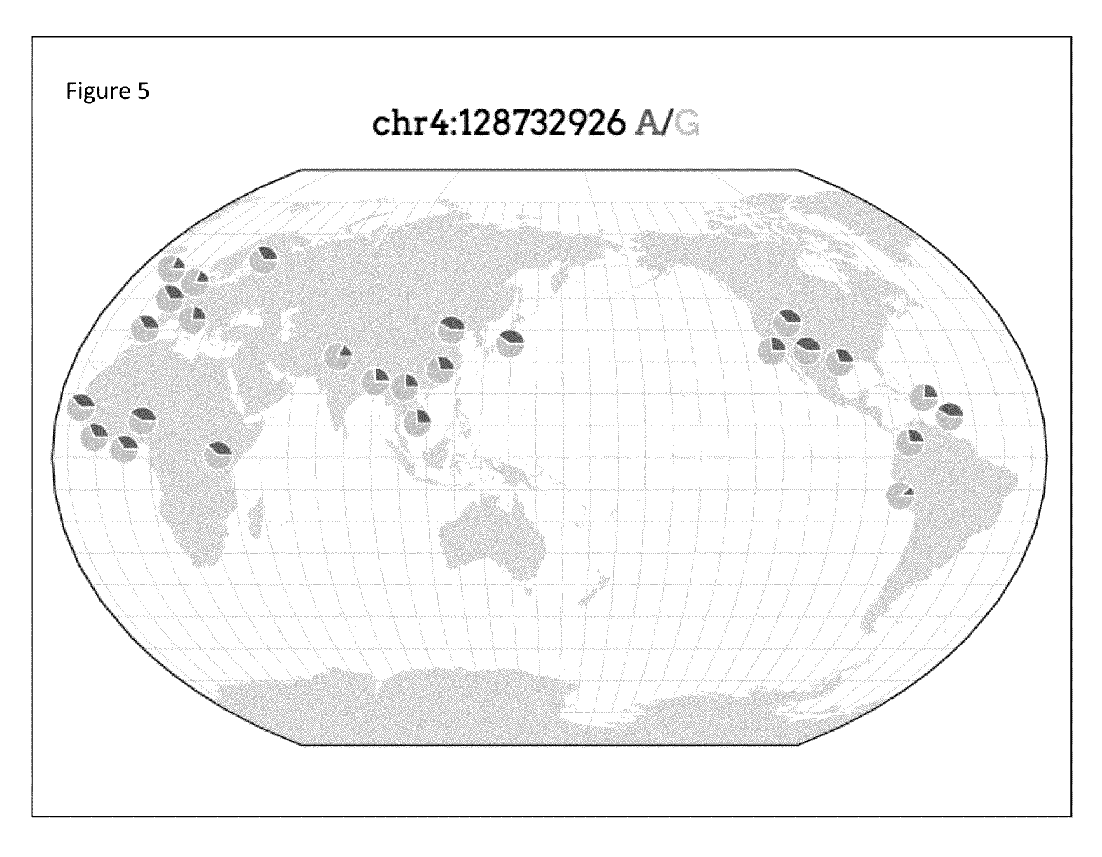

A peak on chromosome 4, regions q28.1-q28.2, was observed that strongly associated with the mitotic-error phenotype (FIG. 3C-E). Genotyped SNP rs2305957 was most strongly associated, with the minor allele conferring a significantly increased rate of mitotic error (β=0.218, SE=0.0270, P=8.68×10⁻¹⁶). The minor allele is extremely common, present in diverse human populations at frequencies of 20%-45% (FIG. 5) (1000 Genomes Project Consortium). As mild genomic inflation was observed for this set of association tests (lambda=1.059), all P-values were adjusted using the genomic control approach (Devlin, 1999), resulting in a corrected P-value of 5.99×10⁻¹⁵. No significant associations between paternal genotype and the same mitotic-error phenotype (P=0.389) were observed, which effectively served as a negative control to demonstrate that population stratification did not drive the significant association with maternal genotype (FIGS. 3A & B). The observed association proved robust when separately tested for mothers of European and East Asian ancestries (see Table 1). No additional variants achieved genome-wide significance when controlling for genotype at rs2305957, thereby providing no evidence of epistatic interactions with this locus.

TABLE 1
Association between genotype and the same mitotic-error phenotype for
a subset of 1,332 female patients of European and East Asian ancestry.

| | Sample size | | | | | | Uncorrected | | Genetic control |
|---|---|---|---|---|---|---|---|---|---|
| | Patients | Embryos | β | SE | OR | 95% CI | λ | P | P |
| Discovery | 2,362 | 20,798 | 0.218 | 0.027 | 1.244 | (1.179--1.311) | 1.059 | 8.68E−16 | 5.99E−15 |
| Europe | 1,332 | 11,861 | 0.214 | 0.0353 | 1.238 | (1.155--1.327) | 1.066 | 1.91E−09 | 6.67E−09 |
| East Asia | 259 | 2,222 | 0.28 | 0.0788 | 1.323 | (1.133--1.543) | 1.088 | 4.58E−04 | 8.51E−04 |
| Validation | 34 | 283 | 0.589 | 0.219 | 1.802 | (1.173--2.768) | NA | 0.0112 | NA |

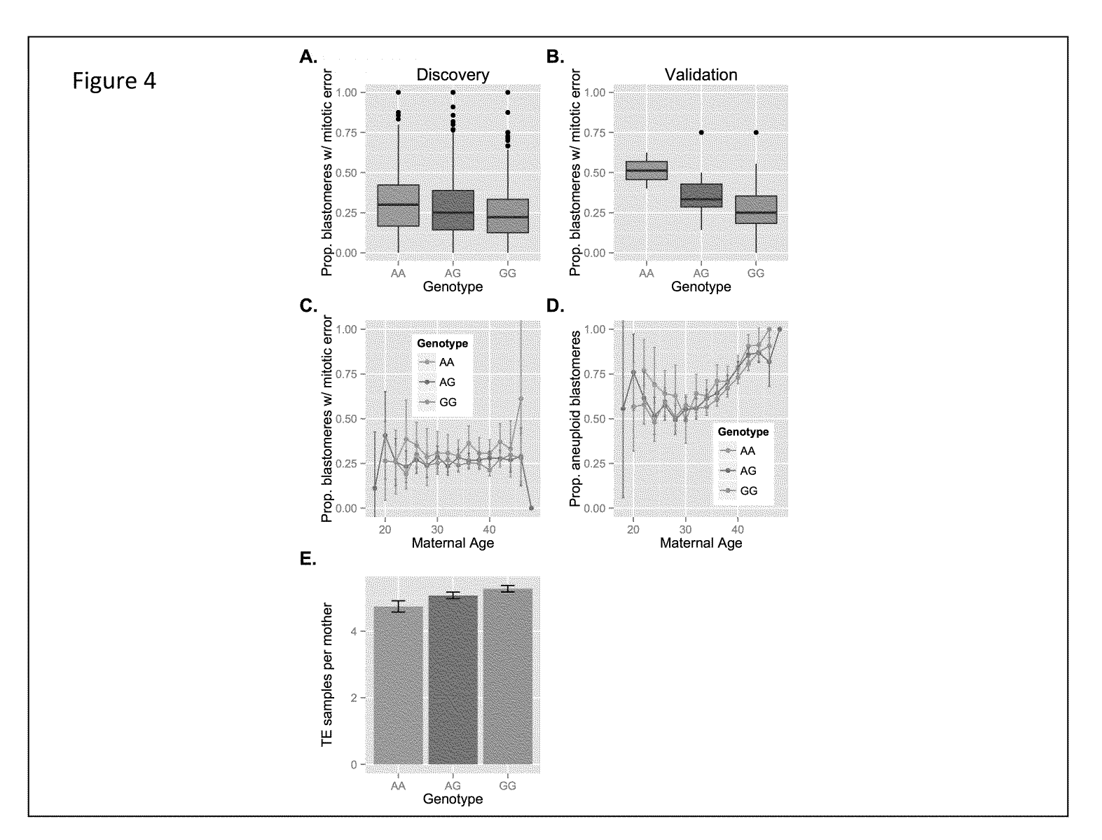

The observed effect size was substantial, with means of 24.6%, 27.0%, and 31.7% of blastomeres affected with paternal-chromosome aneuploidies for the ‘GG’, ‘AG’, and ‘AA’ maternal genotypic classes, respectively (FIG. 4C). The effect was consistent across age classes, suggesting no interaction with maternal age (FIG. 4D). We additionally note that the effect size based on individual blastomeres may underestimate the overall effect on aneuploidy, as diploid blastomeres will be sampled by chance from some diploid-aneuploid mosaics. The frequencies of the three genotypes were not significantly different, however, between mothers and fathers or between egg-donors and non-donors, together suggesting that this set of IVF patients is not enriched for the mitotic-error-associated genotypes.

For validation, genotypes from 34 additional unrelated mothers were tested for association with the same phenotype. Despite the small sample size (34 patients, 283 blastomeres), the association was replicated in this independent sample, with 25.3%, 35.7%, and 51.3% of blastomeres with errors affecting paternal chromosomes among the three respective maternal genotypic classes (B=0.589, SE=0.219, P=0.0112; FIG. 4B).

By initially limiting the phenotype to blastomeres with aneuploidies affecting paternal chromosomes, a subset of aneuploidies was identified that was likely to have been generated during post-zygotic cell divisions. As previously mentioned, however, errors affecting paternal chromosomes commonly co-occur with other forms of aneuploidy, especially in the case of paternal chromosome loss. We were therefore interested in whether other phenotypes characteristic of post-zygotic errors, namely complex aneuploidies involving chromosome losses, were also associated with the same genotype. Upon testing for association with alternative phenotypes, the initial association was found to be driven by aneuploidies that included a paternal chromosome loss (13=0.237, SE=0.0285, P=6.76×10⁻¹⁷), but not those including paternal chromosome gains upon excluding co-occurring cases of chromosome loss (β=0.0198, SE=0.0639, P=0.757). Because mitotic errors are equally likely to affect maternal chromosomes, it was presumed that the association might also be observed for these aneuploidies despite the additional noise due to the prevalence of maternal meiotic error. As the initial association was predominantly driven by chromosome losses, the phenotype was restricted to maternal chromosome loss. Therefore all blastomeres with at least one paternal chromosome loss (rather than including these as controls) were removed from the dataset and tested for an association of rs2305957 genotype with maternal chromosome loss for the remaining 19,576 blastomeres. The independent association was significant in the same direction as the initial association (13=0.0783, 5E=0.0314, P=0.0128), and thereby provided internal validation of the association result.

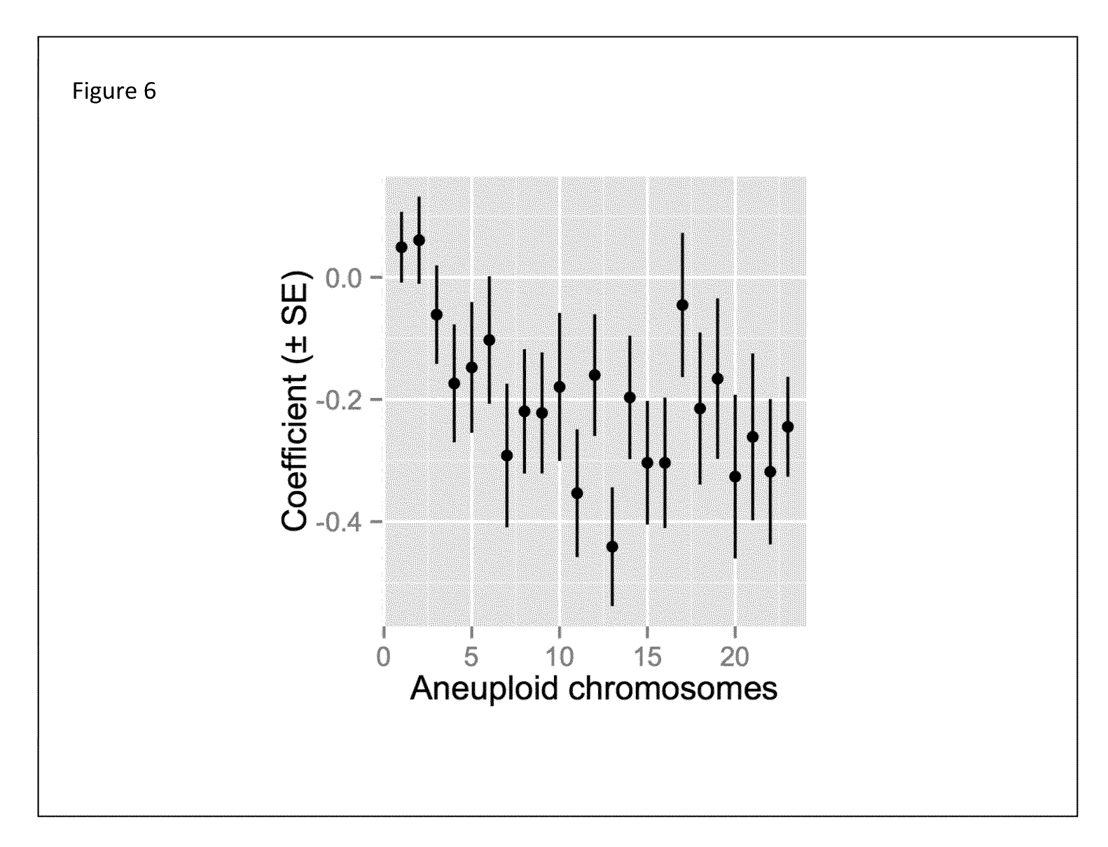

Highlighting its importance, genotype at rs2305957 was also a significant predictor of overall aneuploidy (β=0.156, SE=0.0272, P=8.96×10⁻⁹; FIG. 4D), especially when restricting to complex aneuploidies affecting greater than two chromosomes (β=0.234, SE=0.0329, P=1.72×10⁻¹², FIG. 6). Means of 65.2%, 68.3%, and 71.4% of blastomeres per case were determined to be aneuploid for mothers with the ‘GG’, ‘AG’, and ‘AA’ genotypes, respectively. This 5.3% difference in proportion of aneuploid blastomeres between the two homozygous maternal genotype classes was roughly equivalent to the average effect of 1.8 years of age for mothers ≧35 years old (FIG. 7).

Given that the reported association was driven by complex aneuploidies affecting many chromosomes, and that complex and mosaic aneuploidies are more likely to be inviable (Vega, 2014), in a next step it was tested whether the arrest of aneuploid embryos would bias the genotypic ratios at associated SNPs for embryos sampled at the day-5 blastocyst stage. Herefore, 15,388 trophectoderm biopsies were investigated which were sampled from IVF cycles of 2,998 unrelated mothers who were additionally genotyped on the same SNP microarray. Individuals with the mitotic error-associated genotypes at rs2305957 contributed significantly fewer trophectoderm biopsies for testing (β=−0.0619, SE=0.0204, P=0.00247, FIG. 4E), consistent with an increased proportion of inviable aneuploidies.

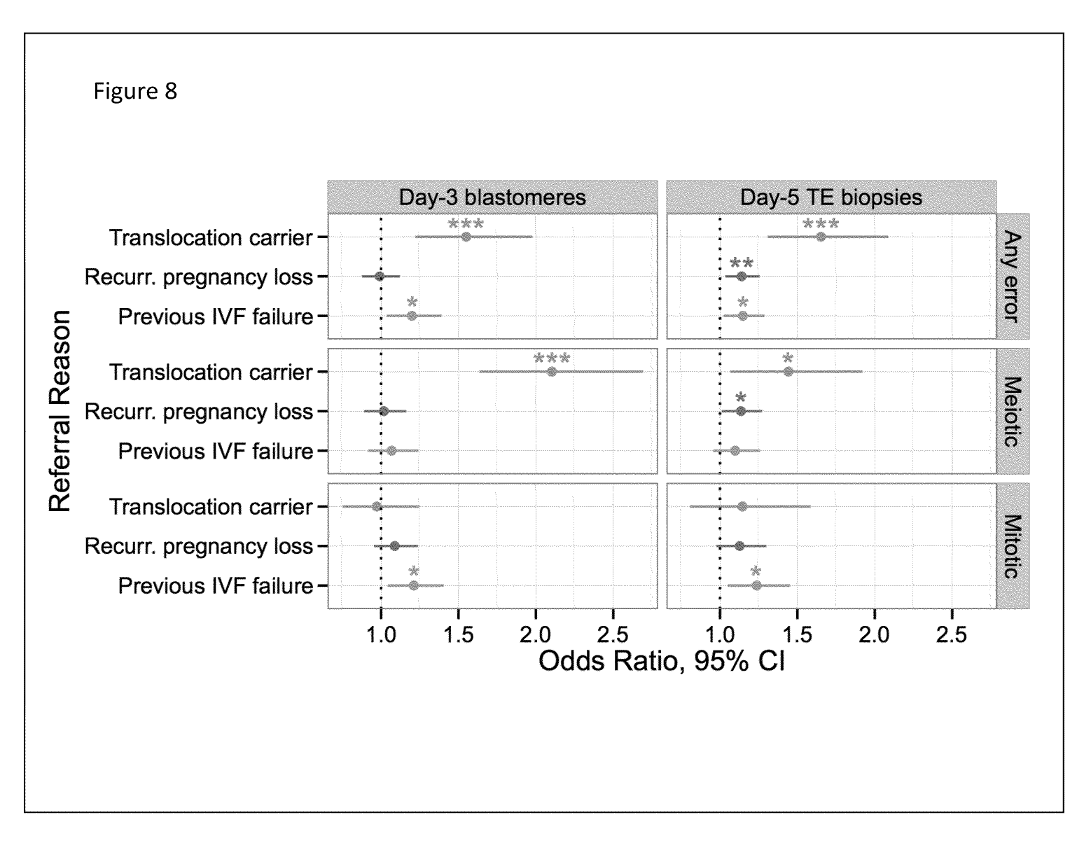

Observed rates of meiotic and mitotic errors were tested against the reasons for referral to preimplantation genetic screening; the effect of maternal age was regressed out where appropriate. Known carriers of translocations had significantly higher rates of meiotic errors than patients referred for other reasons. Patients with previous IVF failure had higher rates of mitotic error compared to patients with recurring pregnancy loss (FIG. 8). The observation of higher mitotic error rates also suggests that an increase in mitotic error may negatively affect fertility and it may take longer, on average, for women with genotypes that are associated with higher mitotic error to achieve successful pregnancies.

In order to characterize the extent of the associated region, genotype imputation for a subset of 1,332 patients of European ancestry was performed (see Experimental Procedures, above). The associated haplotype lies in a region of low recombination and spans greater than 600 Kbp of chromosome 4, regions q28.1-q28.2 (FIG. 3E), including genes INTU, SLC25A31, HSPA4L, PLK4, MFSD8, LARP1B, and PGRMC2. Among this set, the ciliogenesis-related gene INTU and the progesterone receptor PGRMC2 were possible causal candidates, the latter implicated as a potential tumour supressor (Wendler & Wehling, 2013). The gene PLK4, however, stands out as the leading candidate based on its well-characterized role as the master regulator of centriole duplication, a key component of the centrosome cycle (Habedanck et al., 2005, Bettencourt et al., 2005). In addition, it was recently demonstrated that PLK4 was essential for mediating bipolar spindle formation during the first cell divisions in mouse embryos which take place in the absence of centrioles (Coelho et al., 2013).

Due in part to the observation that centrosome aberrations and aneuploidies are common in human cancers, the role of PLK4 and its orthologs in mediating the centrosome cycle has been extensively investigated in several model systems. PLK4 is a tightly-regulated, low-abundance kinase with a short half-life (Firat et al., 2014). Overexpression of PLK4 results in centriole overduplication, thereby increasing the frequency of multipolar spindle formation and subsequent anaphase lag (Ganem et al., 2009). Anaphase lag is a common mechanism contributing to aneuploidy in early embryos and results in chromosome loss with no corresponding chromosome gain (Coonen et al., 2004), consistent with the association signature observed herein. Reduced expression of PLK4 resulted in centriole loss (Bettencourt et al., 2005), which also leads to multipolar spindle formation, as well as the formation of monopolar spindles. Both up- and down-regulation of PLK4 therefore have the potential to induce chromosome instability, and altered PLK4 expression is commonly observed in several forms of cancer, consistent with a tumor-supressor function (Ko et al., 2005).

Along with hundreds of variants upstream and downstream of PLK4, the associated region contains two nonsynonymous SNPs within the PLK4 coding sequence: rs3811740 (S232T) and rs17012739 (E830D), the former occurring in the protein's kinase domain and the latter occurring in the crypto Polo-box domain (Silliboume et al., 2010). Neither site exhibits strong conservation over deep evolutionary time, and both SNPs were predicted as benign based on sequence conservation, amino acid similarity, and mapping to three-dimensional protein structure (see Experimental Procedures, above).

Based on the observation that the minor allele of SNP rs2305957 is derived and segregates at intermediate frequencies in diverse human populations yet is absent from Neanderthal (Green et al., 2010) and Denisovan (Meyer et al., 2012) genomes, it was investigated in a further step whether the region showed evidence of positive selection in ancient human history. Unfortunately, signatures detectable by classic frequency spectrum-based tests decay on the order of IVF, generations, and thus capture only relatively recent human evolutionary history. Green et al. (Green et al., 2010) however, devised an approach with unique resolution to detect signatures of ancient selective sweeps. They first computed the probability that human SNPs at various derived allele frequencies would also be observed in the Neanderthal genome. Deficiencies compared to this expectation, when occurring over large regions, served as evidence of strong selective sweeps occurring in ancient humans after divergence from the Neanderthal lineage. The mitotic-error associated region identified in our study is among the 212 previously-identified regions displaying such a signature. This finding suggests that either this seemingly deleterious variation hitchhiked to high frequency with a linked adaptive variant or that the causal variant was itself adaptive in a context that is not currently understood.

In summary, it is shown that mitotic fidelity is affected by variation in maternal gene products controlling the initial cell divisions of preimplantation embryos. This finding is important in the context of IVF, where selection of euploid embryos may improve the success rate of implantation and ongoing pregnancy (Scott et al., 2013, Forman et al., 2013). More broadly, factors influencing variation in rates of aneuploidy may also help explain variation in fertility status among the general population. Only 30% of all human conceptions result in successful pregnancy, a fact which is mostly explained by high rates of inviable aneuploidy in early development (Baart & Van Opstal 2014). The identification of genetic variation influencing rates of aneuploidy is an important step in the understanding of aneuploidy risk and may assist the future development of diagnostic or therapeutic technologies targeting certain forms of infertility.

### 8.2 Example 2

### Chromosome-Specific Pattern of Overall Aneuploidy

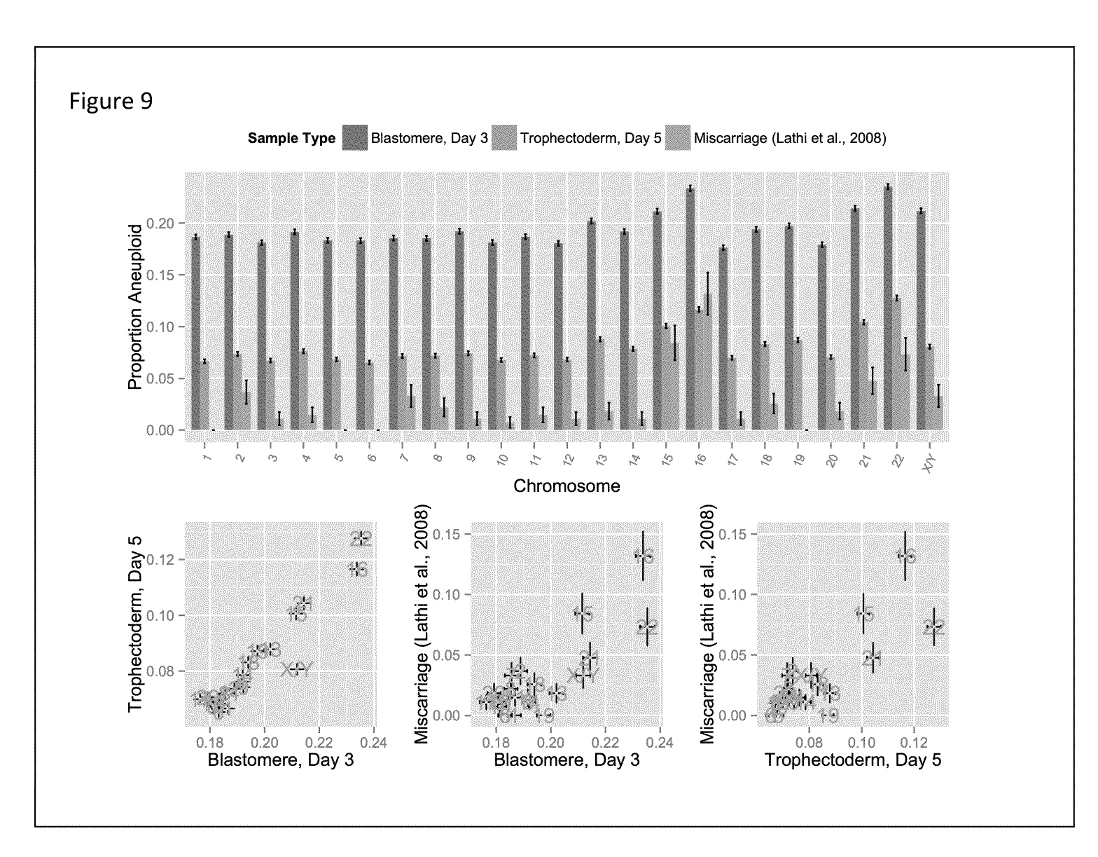

Aneuploidy does not affect all chromosomes equally. Among blastomere samples, per-chromosome rates of whole-chromosome aneuploidy were found to range from 17.6% for chromosome 17 to 23.5% for chromosome 22, with chromosome 16 (23.4%), chromosome 21 (21.4%), the sex chromosomes (21.2%), and chromosome 15 (21.2%) being the next most commonly affected (see FIG. 9A). Chromosomes 15, 16, 21 and 22 also had the highest rates of aneuploidy in day-5 trophectoderm biopsies (10.1%-12.8%). Notably, aneuploidies of these same chromosomes were enriched among products of conception (4.8%-13.2%) in a previous study of 273 miscarriages diagnosed by ultrasound between 6 and 10 weeks of gestation (Lathi et al., 2008). Several other studies of spontaneous abortions (Ljunger et al., 2005; Yusuf et al., 2004; Rolnik et al., 2010; Menasha et al., 2005; Nagaishi et al., 2004) obtained qualitatively similar results, with trisomy 16 being the most common form of aneuploidy in every study.

A strong correlation of per-chromosome rates of aneuploidy was observed between blastomere and trophectoderm samples (r=0.954, P<1×10⁻¹⁰, see FIG. 9B). Per-chromosome rates of aneuploidy in the aforementioned study of miscarriages (Lathi et al., 2008) were also strongly correlated with the observed blastomere (r=0.823, P=1.43×10⁻⁶, see FIG. 9C) and trophectoderm biopsy data (r=0.821, P=1.60×10⁻⁶, see FIG. 9D) despite the difference in sample size. Thus, elevated aneuploidy incidence for particular chromosomes is a feature that appears independent of the time point at which sampling occurs.

Certain chromosomal signatures are highly indicative of meiotic versus mitotic error, while other signatures could arise via either process. To learn about chromosome-specific patterns of various aneuploidy-generating mechanisms different forms of aneuploidy were separately investigated based on informative signatures.

Both Parental Homologs Aneuploidies.

One informative signature is the presence of homologs from either both maternal grandparents or both paternal grandparents in a single region of the embryo's genome. These unique cases of chromosome gain, termed herein ‘both parental homologs’ (BPH) aneuploidies, are very likely meiotic rather than mitotic in origin, as isolated mitotic errors cannot produce this outcome. Chromosome gains were alternatively designated as ‘single parental homolog’ (SPH) aneuploidies if two of the homologs were inferred to be identical along their entire length. SPH aneuploidies cannot be unequivocally assigned to mitotic errors, however, as meiosis II errors in the absence of recombination can also result in SPH trisomy (see Rabinowitz et al., 2012).

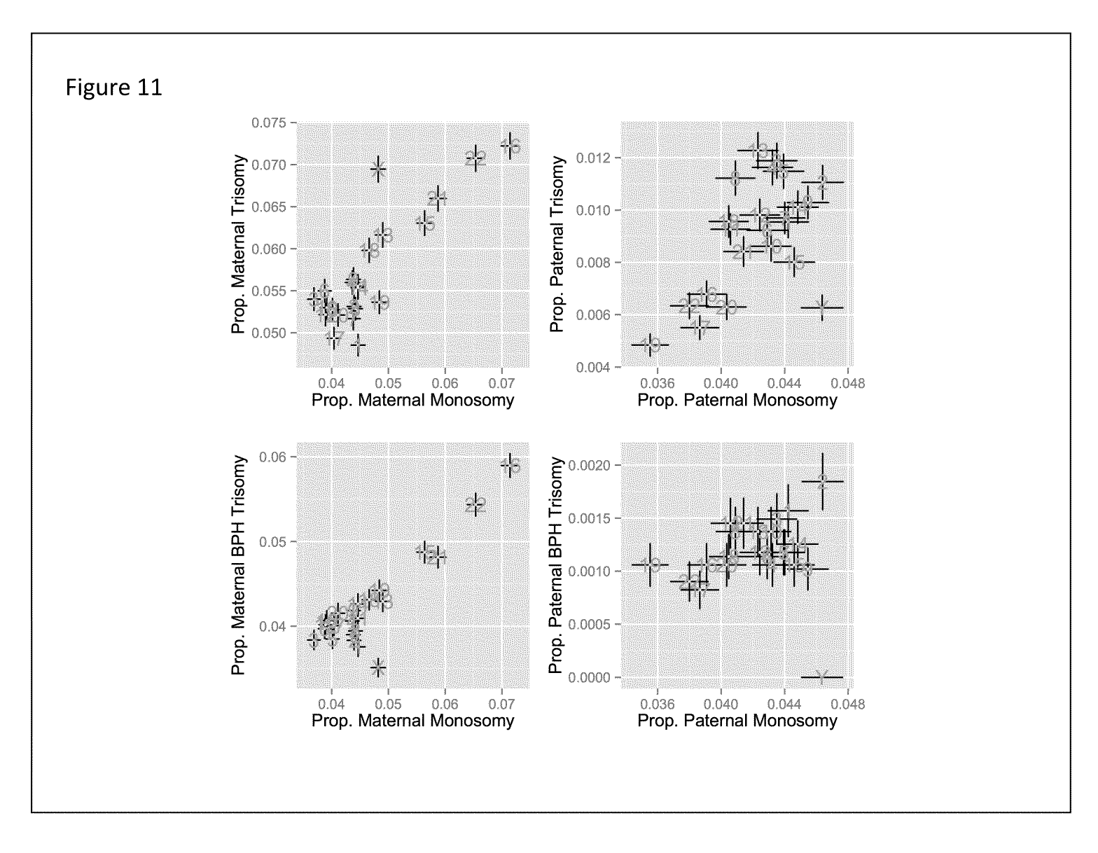

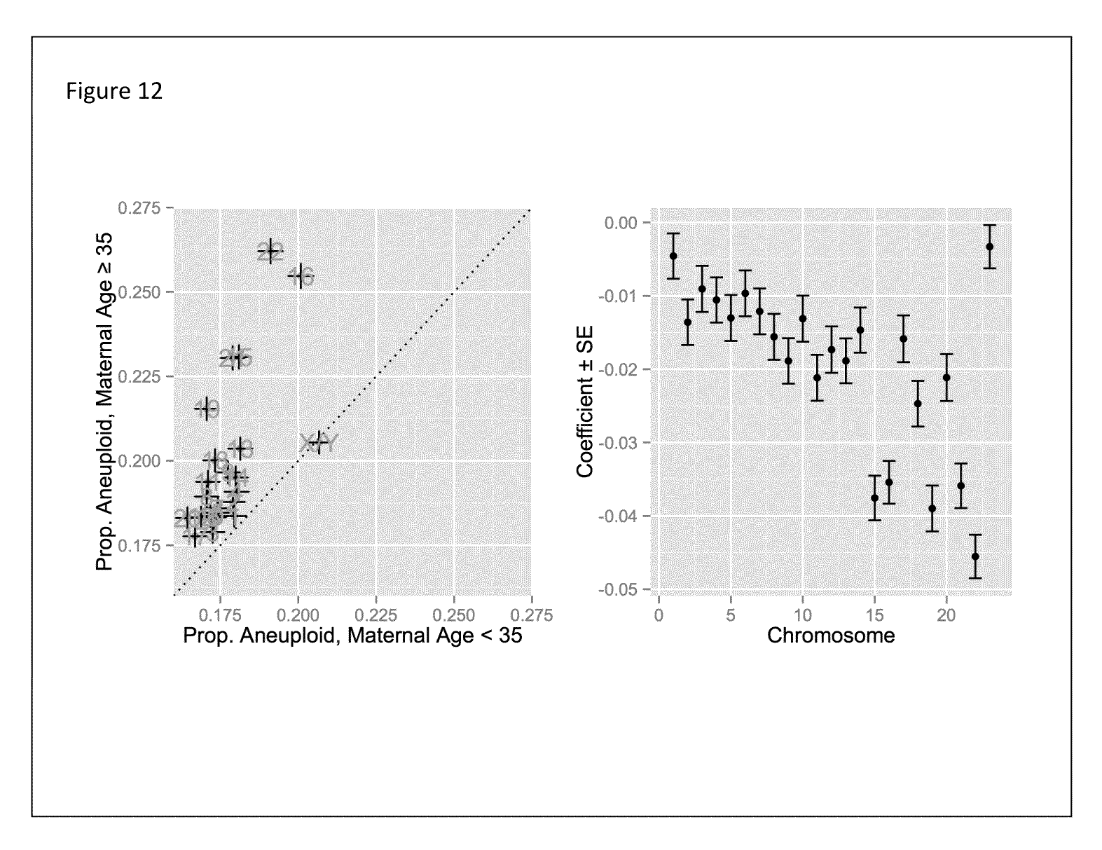

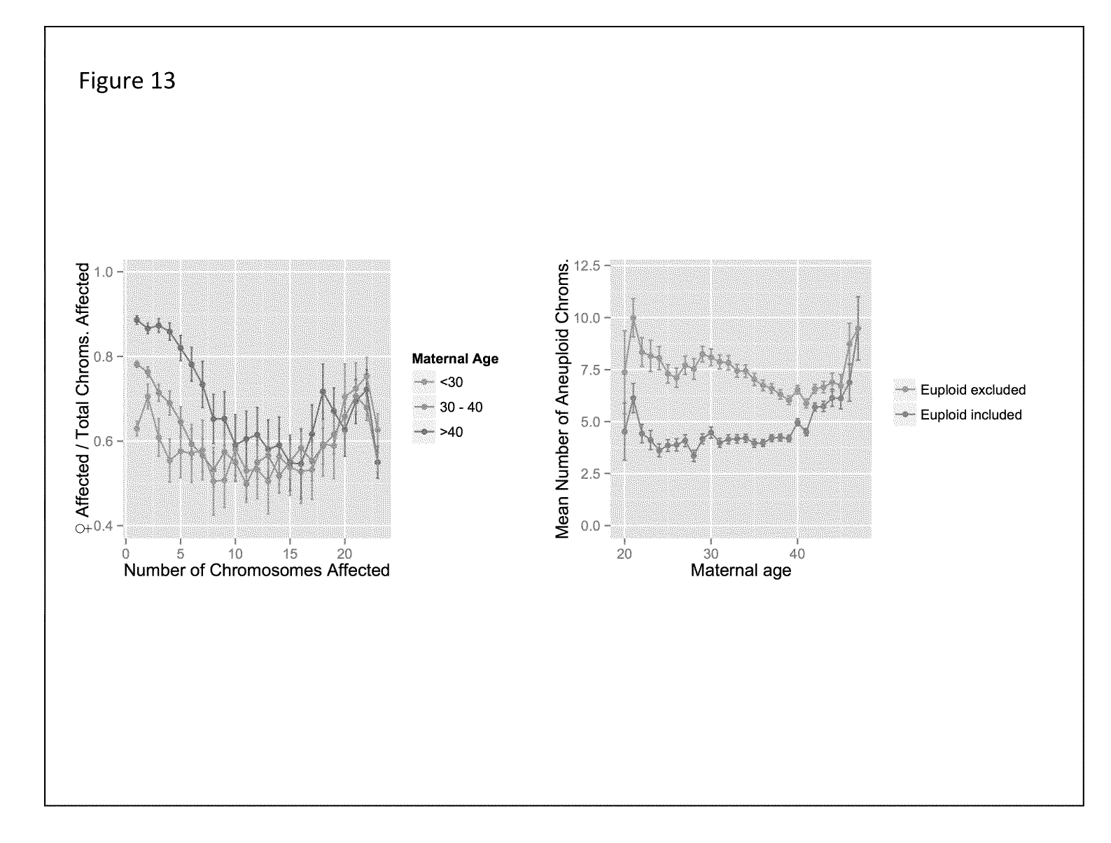

Maternal trisomy and maternal monosomy were found to more often affect smaller chromosomes (Maternal trisomy: r=−0.443, P=0.0343, FIGS. 9A & 9E); Maternal monosomy: r=0.494, P=0.0166, FIGS. 9B & 9F), driving the chromosome-specific profile for overall aneuploidy. Chromosome-specific rates of maternal trisomy and maternal monosomy were also highly correlated with one another (r=0.849, P=2.99×10⁻⁷, see FIG. 11A), an effect which became even stronger when maternal BPH trisomies were separately considered (r=0.897, P=6.98×10⁻⁹, see FIG. 11C), reflecting the fact that many maternal BPH trisomies and maternal monosomies likely share a common origin of meiotic non-disjunction or unbalanced chromatid predivision. In further support, chromosomes 16, 22, 15, and 21, which had the highest rates of maternal trisomy and monosomy, also displayed the strongest increases with maternal age, greatly exceeding the maternal-age effects on other chromosomes (FIGS. 11A & 11B). Chromosome 19 also displayed a strong maternal-age effect despite having a relatively lower rate of aneuploidy, an observation that was nevertheless consistent with the negative correlation between chromosome length and age-associated meiotic-error susceptibility. A generalized linear model confirmed the presence of a length-by-age interaction effect on probability of maternal BPH trisomy affecting particular chromosomes (β=1.636×10⁻¹⁰, SE=2.638×10⁻¹¹, P=1.00×10⁻⁹).

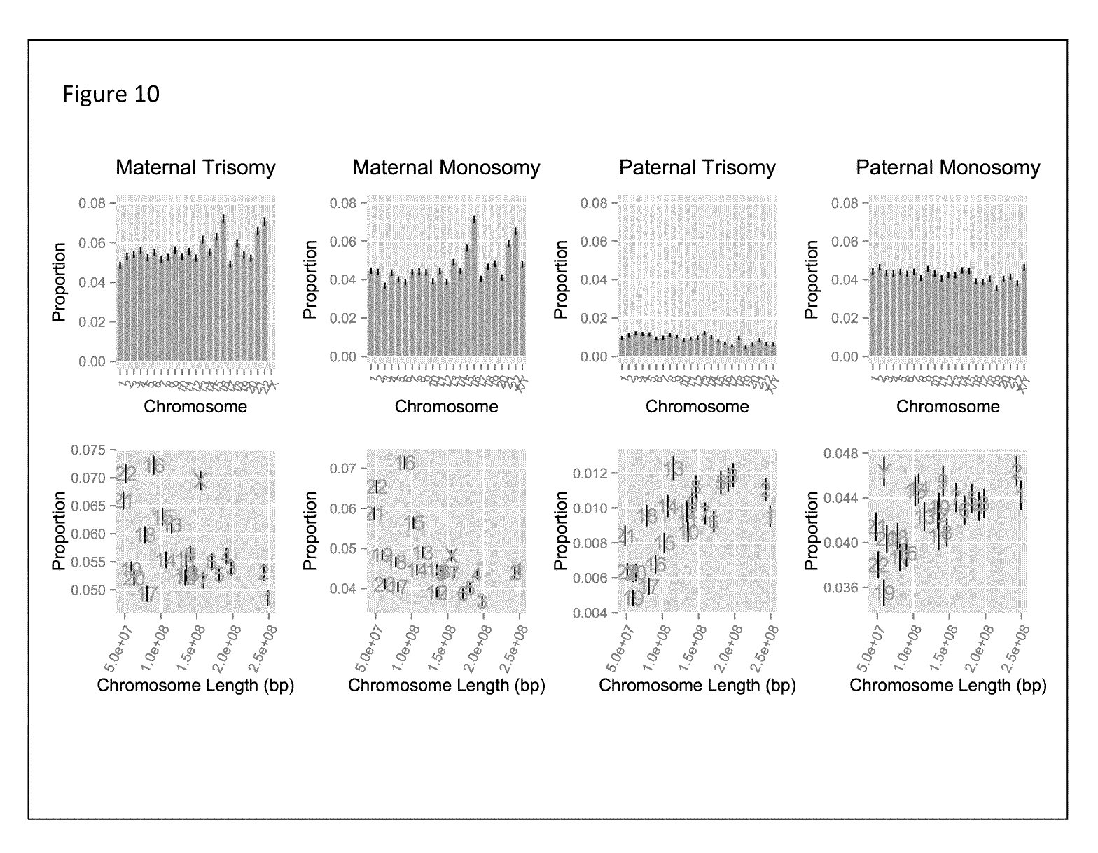

Errors affecting paternal chromosome copies, meanwhile, are good indicators of mitotic-origin aneuploidy. As shown in FIG. 10, rates of paternal trisomy and paternal monosomy were elevated among larger chromosomes (paternal trisomy: r=0.701, P=1.92×10⁻⁴, FIGS. 9C & 9G; paternal monosomy: r=0.560, P=0.00541; FIGS. 9D & 9H). This suggested that while meiotic errors were biased toward smaller chromosomes, mitotic errors displayed the opposite pattern with larger chromosomes more frequently being affected. As shown in FIG. 11, chromosome specific rates of paternal trisomy and paternal monosomy were also correlated (r=0.566, P=0.00491, FIG. 11B), indicating that the chromosome-specific pattern was likely driven by mitotic non-disjunction. No significant correlation was detected between chromosome-specific rates of rare paternal BPH trisomy and relatively common paternal monosomy (r=0.071, P=0.748, FIG. 11D), consistent with the interpretation that paternal meiotic error was not responsible for elevated rates of aneuploidy on particular chromosomes.

Mitotic errors are expected to equally affect maternal and paternal chromosome copies, so we were intrigued by the observation that maternal monosomy (26,261 total chromosomes affected) was only slightly more common than paternal monosomy (24,454 total chromosomes affected) despite a high incidence of maternal BPH trisomy which presumably arises via maternal meiotic error, and thus also produces monosomic daughter cells. We therefore expected maternal monosomies to be more common, as they arise as a consequence of meiotic non-disjunction and chromatid predivision as well as mitotic anaphase lag, all considered common mechanisms of aneuploidy formation. The deficiency of maternal monosomies compared to the expectation suggested early viability selection against monosomic daughter cells following meiotic non-disjunction or chromatid predivision events, a bias which is well-known for later developmental stages (Hassold et al., 1986).

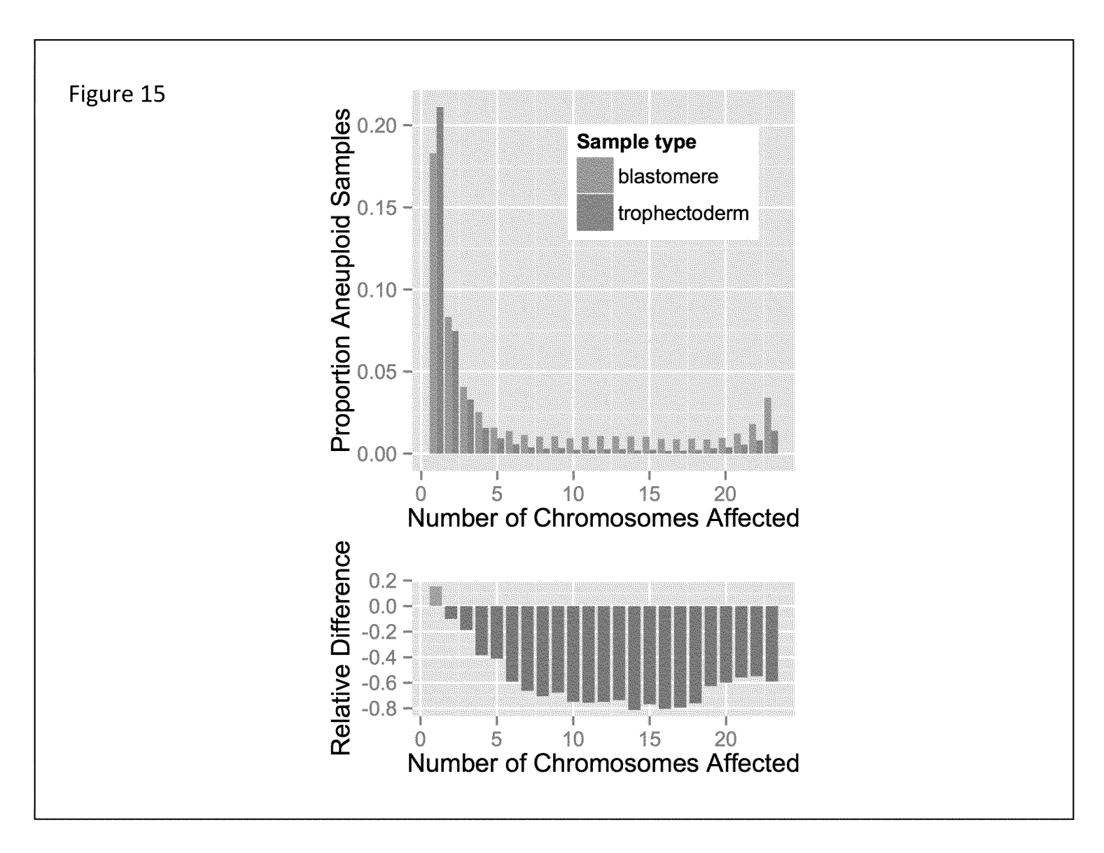

Meiotic errors may also have less influence on chromosome-specific rates of aneuploidy because they tend to affect fewer chromosomes. This trend can be observed by calculating the relative proportions of maternal and paternal chromosomes contributing to aneuploidies with varying numbers of total chromosomes affected (FIG. 15A). Mitotic errors are expected to affect maternal and paternal chromosome copies equally, so this ratio should approach 50% when considering only mitotic-origin aneuploidies but skew toward higher percentages when more maternal meiotic aneuploidies are included. It was found that errors affecting few or nearly all chromosomes were more biased toward maternal chromosome copies, while errors affecting intermediate numbers of chromosomes were more balanced between maternal and paternal homologs (FIG. 15A). This finding suggests that aneuploidies with intermediate numbers of chromosomes affected are predominantly mitotic in origin, while meiotic errors tend to affect few or nearly all chromosomes. Furthermore, this maternal-error bias became stronger with increasing maternal age, such that for patients greater than 40 years of age, more than 80% of aneuploidies affecting one to five chromosomes affected maternal rather than paternal chromosome copies (FIG. 15A). Meanwhile, the average number of aneuploid chromosomes also increased with maternal age, beginning near age 40 (FIG. 15B), replicating recent results of Franasiaket and coworkers (Franasiak et al., 2014). Upon excluding euploid blastomeres from this calculation, however, the mean number of aneuploid chromosomes first decreased with age before increasing (FIG. 15B), supporting the conclusion that mitotic-origin aneuploidies, which comprise a higher proportion of aneuploidies for younger mothers, tend to affect greater numbers of chromosomes.

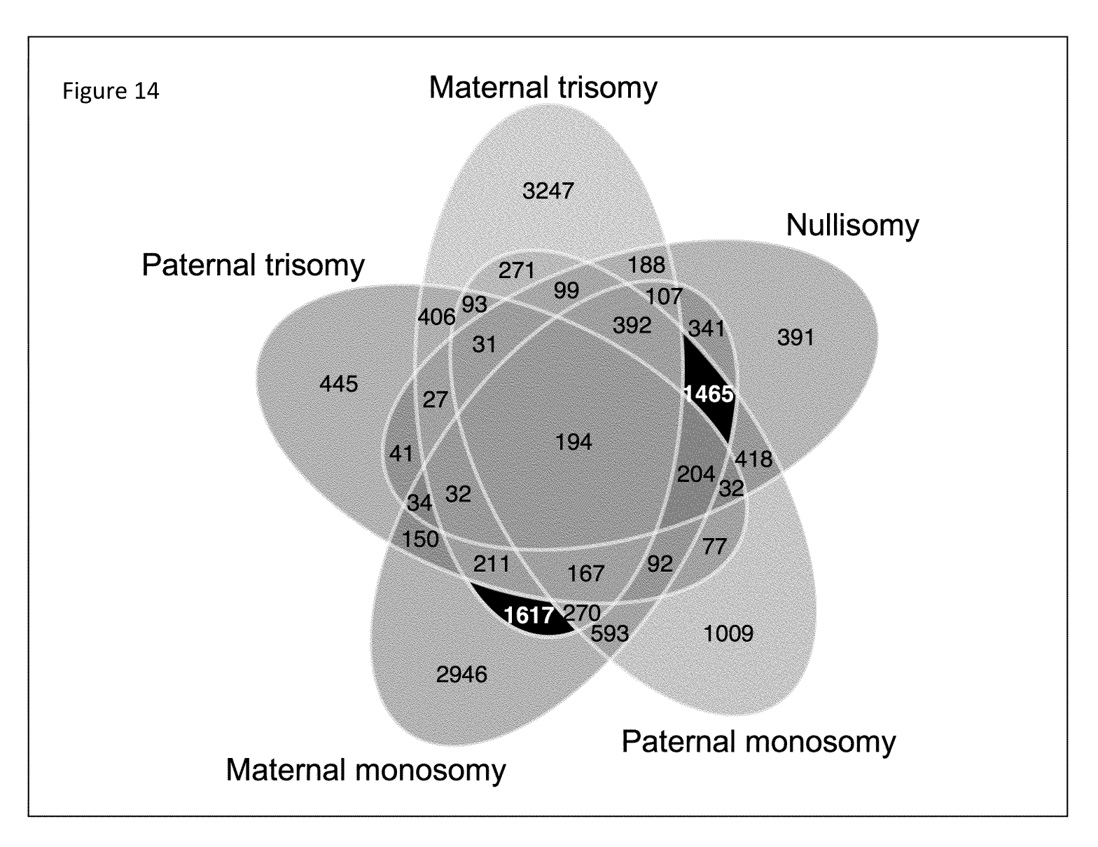

Complex aneuploidies were found non-random in their constitution, with co-occurrence of certain forms of aneuploidy being more common than others (FIG. 14). Maternal monosomy and maternal trisomy, the most prevalent forms of aneuploidy, frequently co-occurred within individual blastomeres. A total of 2,990 blastomeres (11.7%) contained at least one maternal chromosome loss and at least one maternal chromosome gain. Meanwhile, 2,946 blastomeres (11.5%) with maternal chromosome losses and 3,247 blastomeres (12.7%) with maternal chromosome gains occurred in isolation of all other forms of aneuploidy. A second common form of complex aneuploidy involved the co-occurrence of multiple forms of chromosome loss. Maternal monosomy, paternal monosomy, and nullisomy co-occurred in 1,465 individual blastomeres (5.7%). Table 2 shows the rates of various forms of whole-chromosome abnormalities observed in day-3 blastomere biopsies and day-5 trophectoderm (TE) biopsies.

TABLE 2
Rates of various forms of whole-chromosome abnormalities observed in day-3
blastomere biopsies and day-5 TE biopsies. Counts and proportions of total sample
are reported for each sample type. Complex errors involving multiple chromosomes
decrease in frequency between days 3 and 5, while errors of putative meiotic origin
(e.g. maternal BPH trisomy) display a corresponding increase. Maternal triploidies are
defined as containing an extra set of maternal chromosomes. Maternal haploidies are
defined as containing only a maternal set, but no paternal set of chromosomes.
Paternal triploidies and haploidies follow this same naming convention with respect to
paternal chromosome sets. Near-triploidies and near-haploidies
arbitrarily defined as having 20+ extra or missing chromosomes, respectively.

| Class Of Whole-Chromosome | | | | |
|---|---|---|---|---|
| Abnormality | N Blastomeres (% ± SE) | | N TE Biopsies (% ± SE) | |
| Minor aneuploidies | | | | |
| (≦2 chromosomes affected) | | | | |
| Single trisomy | 2013 | (7.90 ± 0.17%) | 1927 | (11.19 ± 0.24%) |
| Single maternal trisomy | 1695 | (6.65 ± 0.16%) | 1606 | (9.33 ± 0.22%) |
| Single maternal BPH trisomy | 1164 | (4.57 ± 0.13%) | 1031 | (5.99 ± 0.18%) |
| Single maternal SPH trisomy | 531 | (2.08 ± 0.09%) | 575 | (3.34 ± 0.14%) |
| Single paternal trisomy | 318 | (1.25 ± 0.07%) | 321 | (1.86 ± 0.10%) |
| Single paternal BPH trisomy | 41 | (0.16 ± 0.03%) | 45 | (0.26 ± 0.04%) |
| Single paternal SPH trisomy | 277 | (1.11 ± 0.06%) | 276 | (1.60 ± 0.10%) |
| Single monosomy | 2720 | (10.67 ± 0.19%) | 1838 | (10.67 ± 0.24%) |
| Single maternal monosomy | 2088 | (8.19 ± 0.17%) | 1565 | (9.09 ± 0.22%) |
| Single paternal monosomy | 632 | (2.48 ± 0.10%) | 273 | (1.59 ± 0.10%) |
| Single nullisomy | 153 | (0.60 ± 0.05%) | 34 | (0.20 ± 0.03%) |
| Double error | 2334 | (9.15 ± 0.18%) | 1376 | (7.99 ± 0.21%) |
| Errors of ploidy | | | | |
| (20+ chromosomes affected) | | | | |
| Triploidy/near-triploidy | 751 | (2.95 ± 0.11%) | 295 | (1.71 ± 0.10%) |
| Maternal (digynic) triploidy/ | 725 | (2.84 ± 0.10%) | 271 | (1.57 ± 0.09%) |
| near-triploidy | | | | |
| Paternal (diandric) triploidy/ | 23 | (0.09 ± 0.02%) | 22 | (0.13 ± 0.03%) |
| near-triploidy | | | | |
| Haploidy/near-haploidy | 306 | (1.20 ± 0.07%) | 98 | (0.57 ± 0.06%) |
| Maternal haploidy/near- | 228 | (0.89 ± 0.06%) | 80 | (0.46 ± 0.05%) |
| haploidy | | | | |
| Paternal haploidy/near- | 72 | (0.28 ± 0.03%) | 18 | (0.10 ± 0.02%) |
| haploidy | | | | |
| Complex errors | | | | |
| 3-19 chromosomes affected | 6304 | (24.72 ± 0.27%) | 1790 | (10.40 ± 0.23%) |

While a plurality of errors affected only one chromosome, greater than 80% of aneuploid blastomeres contained two or more aneuploid chromosomes (FIG. 15A). Aneuploidies affecting between 6 and 20 chromosomes occurred at an approximately constant rate in blastomere samples, but aneuploidies affecting all or nearly all chromosomes were relatively more common (FIG. 15A). Compared to individual day-3 blastomere samples, fewer aneuploidies affecting multiple chromosomes were detected in multi-cell day-5 trophectoderm biopsies (FIG. 15A). The two sample types were compared by calculating the percent difference in rates of aneuploidy between the blastomere and trophectoderm samples, stratifying by the total number of aneuploid chromosomes. This metric reflects the proportion of embryos that were either lost or self-corrected between the two sampling stages. Due to the design of our study, one could not distinguish between selection and self-correction, as blastomere and trophectoderm biopsy data were entirely independent. Nevertheless, we observed that aneuploidies affecting increasing numbers of chromosomes were increasingly depleted in trophectoderm biopsies relative to blastomeres, plateauing at approximately 11 chromosomes affected (FIG. 15B). This difference became less extreme when greater than 15 chromosomes were affected (FIG. 15B).

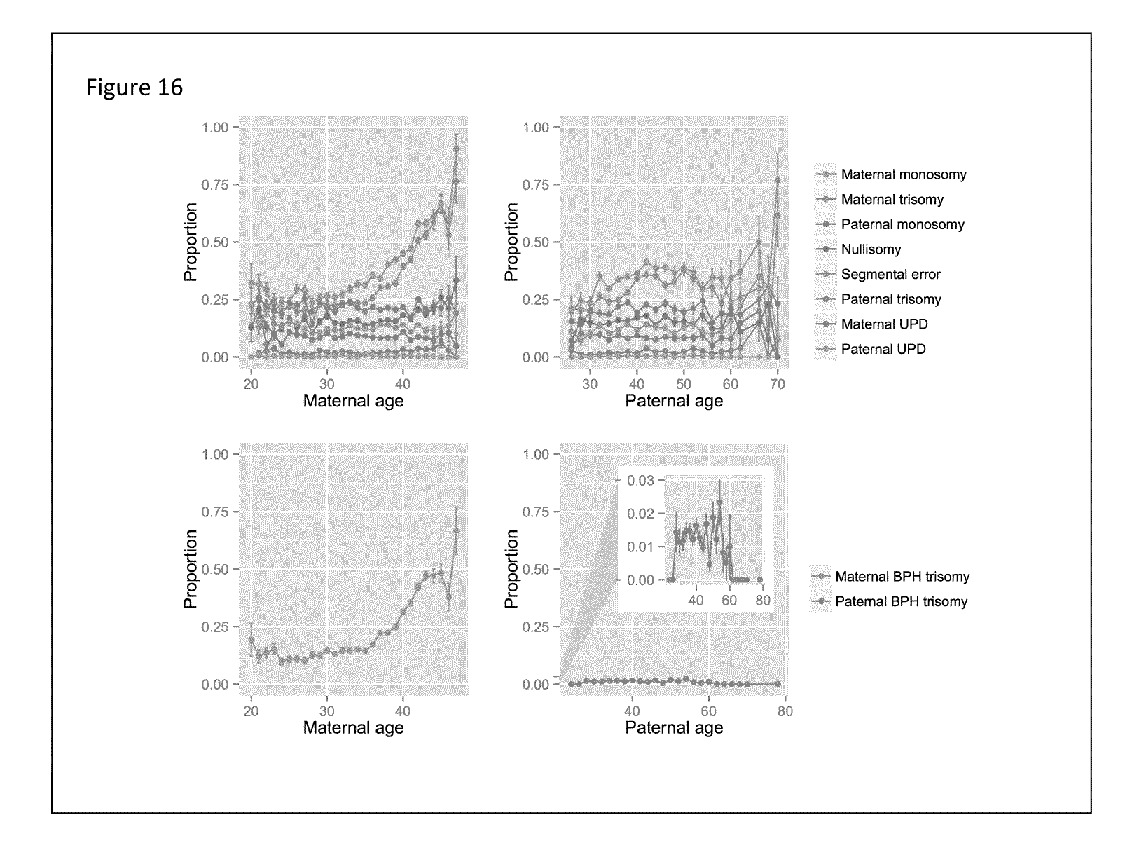

Maternal trisomy (β=0.0785, SE=0.00322, P<1×10⁻¹⁰), maternal monosomy (b=0.0765, SE=0.00307, P<1×10⁻¹⁰ maternal uniparental disomy (β=0.0377, SE=0.00877, P=1.75×10⁻⁵), and nullisomy (β=0.0204, SE=0.00429, P=2.15×10⁻⁶) all significantly increased with maternal age, while errors affecting paternal chromosome copies were not significantly associated with maternal age (β=−0.00210, SE=0.00268, P=0.432; FIG. 16A). The pattern of association between aneuploidy and maternal age was also chromosome-specific (FIG. 14), as has been previously reported based on smaller samples from different developmental time points (Hassold et al., 1986).

To address the question to what extent environmental and/or genetic factors affect rates of aneuploidy, blastomere samples were randomly permuted among families, calculating the across-family variance in proportion of aneuploid blastomeres for each of 500 permutation replicates. This procedure was repeated after matching cases for maternal age, then again after additionally matching cases for paternal age. The resulting variance distributions were then compared to the observed variance in the actual data. The observed variance substantially exceeded the variance attributable to maternal and paternal ages and sampling noise (FIG. 15). Together, these observations provide strong evidence that uncharacterized family-specific factors contribute to variation in rates of aneuploidy.

### Experimental Procedures

Cell Isolation, DNA Amplification, and Genotyping.

Genetic material was obtained from oocyte donors (buccal swabs), fathers (peripheral venipuncture), and embryos (either single-cell day 3 blastomere biopsy or multi-cell day 5 trophectoderm biopsy). Single tissue culture (PMNs) and egg donor buccal cells were isolated using a sterile tip attached to a pipette and stereomicroscope (Leica; Wetzlar, Germany). For fresh day-3 embryo biopsy, individual blastomeres were separated via micromanipulator after zona pellucida drilling with acid Tyrode's solution. A micromanipulator was also used to isolate individual sperm cells. Except for sperm, single cells for analysis were washed four times with buffer (PBS buffer, pH 7.2 (Life Technologies, 1 mg/mL BSA). Multiple displacement amplification (MDA) with proteinase K buffer (PKB) was used for this procedure. Cells were placed in 5 μl PKB (Arcturus PicoPure Lysis Buffer, 100 mM DTT, 187.5 mM KCl, 3.75 mM MgCl₂, 3.75 mM Tris-HCl) incubated at 56° C. for 1 hour, followed by heat inactivation at 95° C. for 10 min, and held at 25° C. for 15 min. MDA reactions were incubated at 30° C. for 2.5 hours and then 65° C. for 10 min. Genomic DNA from buccal tissue was isolated using the QuickExtract DNA Extract Solution (Epicentre; Madison, Wis.). Template controls were included for the amplification method. Amplified single cells and bulk parental tissue were genotyped using the Infinium II (Illumina; San Diego, Calif.) genome-wide SNP arrays (CytoSNP12 chip). The standard Infinium II protocol was used for parent samples (bulk tissue), and Genome Studio was used for allele calling. For single cells, genotyping was accomplished using an Infinium II genotyping protocol.

Aneuploidy Detection.

Detection and classification of various forms of aneuploidy was achieved using the Parental Support algorithm previously described by Johnson et al. (Johnson et al., 2010). This approach uses high-quality genotype data from the father and the mother (or oocyte donor) to infer the presence or absence of homologs in embryo genotype data. This procedure is useful because embryo biopsies incur a high allelic dropout rate due to limited starting material and whole-genome amplification. The procedure was extensively validated in the original publication, with false-positive and false-negative rates that were not significantly different from the ‘gold-standard’ approach of metaphase karyotyping. Furthermore, confidence scores obtained from this approach were strongly correlated with false-detection rates. Treff et al. demonstrated that SNP-microarray-based approaches are more consistent in detecting aneuploidy than widely-used FISH technology (Treff et al., 2010).

Statistical Analyses.

All statistical analyses were conducted using the R statistical computing environment (R Core Team, 2013).

Pearson correlations were used to assess the relationships between chromosome-specific rates of aneuploidy affecting different developmental stages and chromosomes with different lengths as well as the relationship between chromosome-specific rates of different forms of aneuploidy. To test for an interaction between chromosome-specific rates of maternal meiotic aneuploidy and chromosome length, we fit a generalized linear model with the response variable encoded as counts of BPH-aneuploid and non-BPH-aneuploid blastomeres for each chromosome for cases stratified into maternal age groups (rounding to the nearest year). Predictor variables included maternal age, chromosome length, and an interaction of age and chromosome length. The model assumed a binomial error distribution with a logit length. In order to model overdispersion, we did not fix the dispersion parameter (i.e., quasi-binomial).

We also used generalized linear models to test for associations between maternal and paternal ages and various forms of aneuploidy. For each couple, we counted the number of embryos in which a particular form of aneuploidy was detected, while considering all other embryos as controls. We then tested for an association with maternal (or paternal) age using a model that assumed a binomial error distribution with a logit link, again accounting for overdispersion by allowing the dispersion parameter to vary.

To test for an association between aneuploidy incidence and paternal age, the effect of maternal age was controlled using two separate approaches. First, the R package ‘Matching’ (Matching, 2011) was used to sample age-matched mothers from the younger (<$40 years old and older (≧40 years old) paternal age distributions, dropping cases where no match could be achieved within 0.1 standard deviations of the mean maternal age. A 2-by-2 chi-squared contingency table analysis was then employed to contrast counts of aneuploid and euploid blastomeres between the paternal age groups. The other approach was a partial Spearman rank correlation implemented with the R package ‘ppcor’ (Ppcor, 2012), testing for association between paternal age and aneuploidy incidence while holding maternal age constant for both variables.

To calculate percent difference in rates of aneuploidy for the two different sample types, aneuploidies were stratified by the total number of aneuploid chromosomes, followed by the formula:

Percent diff.=Prop.aneuploid blastomeres−Prop. aneuploid trophectoderm biopsies/Prop. aneuploid blastomeres).

In order to demonstrate the existence of an uncharacterized family-specific effect in addition to the effect maternal age, repeat cases from mothers who underwent multiple IVF cycles with PGS by removing all but the first cycle for duplicate (i.e., first-degree relatedness) maternal genotypes using the program KING (Manichaikul et al., 2010). This step reduced the data from 2909 to 2202 cases with at least one blastomere meeting quality-control thresholds and for which maternal age was reported.

Blastomere ploidy status among families was then randomly permuted without replacement, calculating across-family variance in proportion of aneuploid embryos. The procedure was then repeated controlling for maternal age by only permuting among mothers matched within one year of age. Further for paternal age was controlled with an analogous procedure but matching fathers within three years of age. Each set of permutations was repeated 500 times, then compared to the observed variance in per-family proportion of aneuploid blastomeres.

### 8.3. Example 3

### Prediction of Aneuploidy

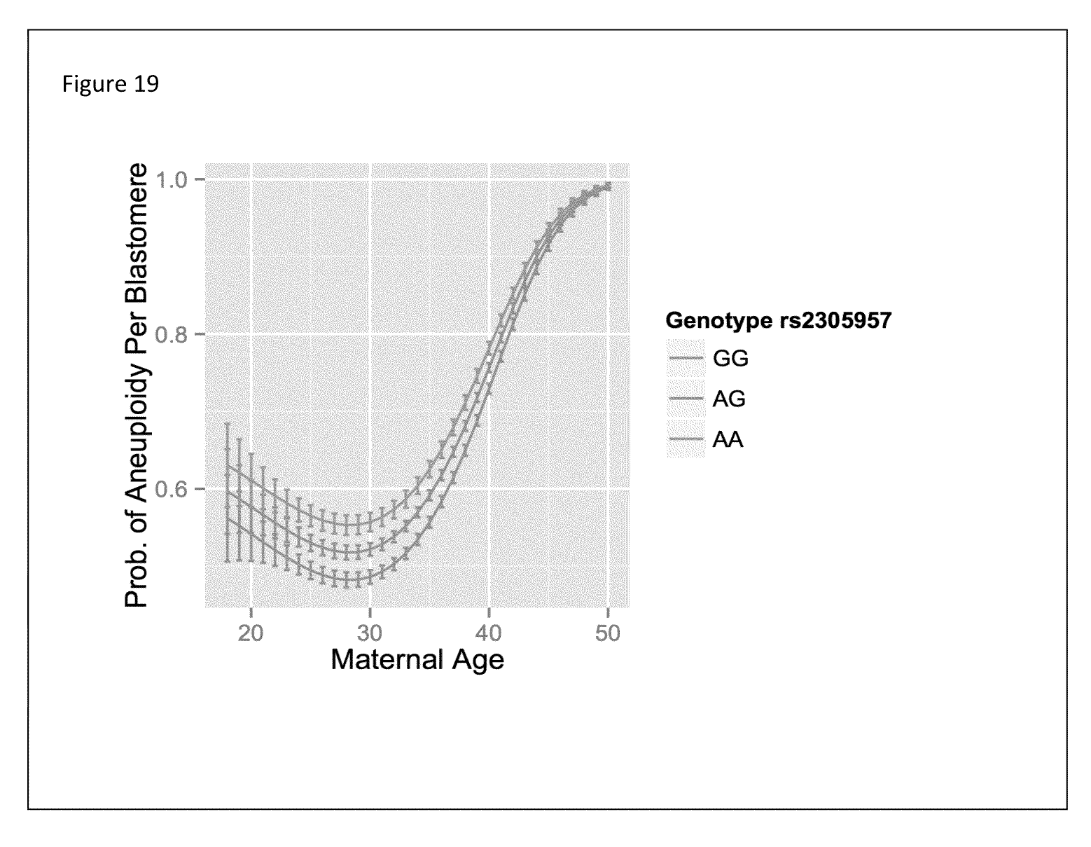

A model was built using data from 1095 unrelated patients: logit(Y)=b₀+b₁X₁+b₂X₁²+b₃X₁³+b₄X₂+ where Y is a two-column matrix containing the counts of euploid and aneuploid blastomeres for each mother, X₁ is the maternal age, and X₂ is the maternal genotype, encoded as the number of alternative alleles at SNP rs2305957 (and linked SNPs in the region of PLK4). Both age and genotype are significant predictors of rate of aneuploidy, together explaining 27% of the variance in proportion of aneuploidy per mother (McFadden's pseudo-R²=0.269). For new cases, we can then use regression prediction methods (such as those implemented using the predict.glm function in R) to estimate the probability of aneuploid conception, along with standard error in the estimate based on predictor variables (see FIGS. 18 and 19). To verify the predictive power of our model, a set of an additional 1095 unrelated patients was used whose embryos were screened for aneuploidy using the same procedure. The Pearson correlation between predicted and observed proportions of aneuploid blastomeres per case was highly significant (r=0.436, P<1×10⁻¹⁰), and even stronger when weighting the correlation by sample size (r=0.516). If the mitotic-error associated genotypes are also associated with increased risk of pregnancy loss and other fertility-related phenotypes, we can then translate the predicted probability of mitotic-error per blastomere into predictions of the probability of inviable aneuploidy, probability of various viable aneuploidies (e.g. Down syndrome, Edwards syndrome), probability of IVF success, the number of embryos required for a high and/or prespecified probability of IVF success, probability of miscarriage, average time to successful conception with unprotected intercourse timed near ovulation, and other aneuploidy-associated fertility outcomes (see FIGS. 18 and 19). These predictions would be achieved using a procedure analogous to the procedure above, but with the response variable being each of these alternative phenotypes.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### 8. REFERENCES

Adzhubei, I. A. et al. A method and server for predicting damaging missense mutations. Nature Methods 7, 248-249 (2010).
Baart, E. B. & Van Opstal, D. Chromosomes in early human embryo. Textbook of Human Reproductive Genetics 52 (2014).
Bettencourt-Dias, M. et al. SAK/PLK4 is required for centriole duplication and flagella development. Current Biology 15, 2199-2207 (2005).
Chelala, C., Khan, A. & Lemoine, N. R. SNPnexus: a web database for functional annotation of newly discovered and public domain single nucleotide polymorphisms. Bioinformatics 25, 655 {661 (2009).
Coelho, P. A. et al. Spindle formation in the mouse embryo requires Plk4 in the absence of centrioles. Developmental Cell 27, 586-597 (2013).
Coonen, E. et al. Anaphase lagging mainly explains chromosomal mosaicism in human preimplantation embryos. Human Reproduction 19, 316-324 (2004).
Cupisti, S. et al. Sequential FISH analysis of oocytes and polar bodies reveals aneuploidy mechanisms. Prenatal Diagnosis 23, 663-668 (2003).
Daphnis, D. et al. Detailed fish analysis of day 5 human embryos reveals the mechanisms leading to mosaic aneuploidy. Human Reproduction 20, 129-137 (2005).
Delhanty, J. D., Harper, J. C., Ao, A., Handyside, A. H. & Winston, R. M. Multicolour FISH detects frequent chromosomal mosaicism and chaotic division in normal preimplantation embryos from fertile patients. Human Genetics 99, 755-760 (1997).
Devlin, B., Roeder, K. & Wasserman, L. Genomic control, a new approach to genetic-based association studies. Theoretical Population Biology 60, 155-166 (2001).
Erickson, J. D. Down syndrome, paternal age, maternal age and birth order. Annals of Human Genetics 41, 289-298 (1978).
Firat-Karalar, E. N. & Stearns, T. The centriole duplication cycle. Philosophical Transactions of the Royal Society B: Biological Sciences 369, 20130460 (2014).
Forman, E. J. et al. In vitro fertilization with single euploid blastocyst transfer: a randomized controlled trial. Fertility and Sterility 100, 100-107 (2013).
Habedanck, R. et al. The polo kinase Plk4 functions in centriole duplication. Nature Cell Biology 7, 1140-1146 (2005).
Hassold, T. & Hunt, P. To en (meiotically) is human: the genesis of human aneuploidy. Nature Reviews Genetics 2, 280-291 (2001).
Hassold, T. & Chiu, D. Maternal age-specific rates of numerical chromosome abnormalities with special reference to trisomy. Human genetics 70, 11-17 (1985).
Howie, B. N., Donnelly, P. & Marchini, J. A exible and accurate genotype imputation method for the next generation of genome-wide association studies. PLoS Genetics 5, e1000529 (2009).
International HapMap 3 Consortium. Integrating common and rare genetic variation in diverse human populations. Nature 467, 52-58 (2010).
Johnson, D. et al. Preclinical validation of a microarray method for full molecular karyotyping of blastomeres in a 24-h protocol. Human Reproduction 25, 1066-1075 (2010).
Ko, M. A. et al. Plk4 haploinsufficiency causes mitotic infidelity and carcinogenesis. Nature Genetics 37, 883-888 (2005).
Kumar, P., Heniko_, S. & Ng, P. C. Predicting the e_ects of coding non-synonymous variants on protein function using the SIFT algorithm. Nature Protocols 4, 1073-1081 (2009).
Manichaikul, A. et al. Robust relationship inference in genome-wide association studies. Bioinformatics 26, 2867 {2873 (2010).
MATLAB. version 7.12.0 (R2011a) (The MathWorks Inc., Natick, Mass., 2011).
Meyer, M. et al. A high-coverage genome sequence from an archaic Denisovan individual. Science 338, 222-226 (2012).
Penrose, L. S. et al. The relative effects of paternal and maternal age in mongolism. J Genet 27, 219-223 (1933).
Purcell, S. et al. PLINK: a tool set for whole-genome association and population-based linkage analyses. The American Journal of Human Genetics 81, 559-575 (2007).
R Core Team. R: A Language and Environment for Statistical Computing. R Foundation for Statistical Computing, Vienna, Austria (2013).
Santos, M. A. et al. The fate of the mosaic embryo: chromosomal constitution and development of day 4, 5 and 8 human embryos. Human Reproduction 25, 1916-1926 (2010).
Scott Jr, R. T. et al. Blastocyst biopsy with comprehensive chromosome screening and fresh embryo transfer signicantly increases in vitro fertilization implantation and delivery rates: a randomized controlled trial. Fertility and Sterility 100, 697-703 (2013).
Sillibourne, J. E. & Bornens, M. Polo-like kinase 4: the odd one out of the family. Cell Division 5, 1-9 (2010).
Simons, Y. B., Turchin, M. C., Pritchard, J. K. & Sella, G. The deleterious mutation load is insensitive to recent population history. Nature Genetics (2014).
Templado, C et al. Aneuploidy in human spermatozoa. Cytogenetic and Genome Research 133, 91-99 (2011).
Turner, S. et al. Quality control procedures for genome-wide association studies. Current Protocols in Human Genetics 1-19 (2011).
Turner, S. D. qqman: an R package for visualizing GWAS results using Q-Q and manhattan plots. bioRxiv (2014).
Voullaire, L., Slater, H., Williamson, R. & Wilton, L. Chromosome analysis of blastomeres from human embryos by using comparative genomic hybridization. Human Genetics 106, 210-217 (2000).
Wells, D. & Delhanty, J. D. Comprehensive chromosomal analysis of human preimplantation embryos using whole genome ampli_cation and single cell comparative genomic hybridization. Molecular Human Reproduction 6, 1055-1062 (2000).
Wendler, A. & Wehling, M. Pgrmc2, a yet uncharacterized protein with potential as tumor suppressor, migration inhibitor, and regulator of cytochrome p450 enzyme activity. Steroids 78, 555-558 (2013).

What is claimed is:

1. A method of determining a woman's susceptibility to conceive an aneuploid embryo, comprising determining the presence or absence of a polymorphic allele in a biological sample from said woman, with the polymorphic allele being in linkage disequilibrium with single nucleotide polymorphism rs2305957 and the polymorphic allele's presence indicating the woman's susceptibility to conceive an aneuploid embryo.

2. The method according to claim 1, wherein the polymorphic allele comprises a single nucleotide polymorphism in linkage disequilibrium with single nucleotide polymorphism rs2305957.

3. The method according to claim 1, wherein the polymorphic allele comprises an insertion or deletion polymorphism in linkage disequilibrium with single nucleotide polymorphism rs2305957.

4. The method according to claim 2, wherein the single nucleotide polymorphism is rs2305957 or a genetic variant in linkage disequilibrium with rs2305957.

5. The method according to claim 1, further comprising
establishing a susceptibility profile by correlating the presence of said polymorphic allele with the woman's age and optionally with prospective father's age; and
comparing said profile to a library of profiles or a prediction model to provide an indication of a woman's susceptibility to conceive an aneuploid embryo,
wherein said comparing provides a determination of said
woman's susceptibility to conceive an aneuploid embryo.

6. A kit for assessing a woman's susceptibility to conceive an aneuploid embryo, the kit comprising reagents for determining the presence or absence of a polymorphic allele in a biological sample from said woman, with the polymorphic allele being located on chromosome 4 and the polymorphic allele's presence indicating the woman's susceptibility to conceive an aneuploid embryo.

7. The kit according to claim 6, wherein the polymorphic allele comprises a single nucleotide polymorphism or in linkage disequilibrium with single nucleotide polymorphism rs2305957.

8. The kit according to claim 6, wherein the polymorphic allele comprises an insertion or deletion polymorphism in linkage disequilibrium with single nucleotide polymorphism rs2305957.

9. The kit according to claim 7, wherein the single nucleotide polymorphism is rs2305957 or a genetic variant in linkage disequilibrium with rs2305957.

10. The kit according to claim 9, further comprising probes that specifically bind to rs2305957.

11. The kit according to claim 6, further comprising instructions to establish a susceptibility profile by correlating the presence of said polymorphic allele with the woman's age and optionally with prospective father's age; and
instructions to compare said profile to a library of profiles known to provide an indication of a woman's susceptibility to conceive an aneuploid embryo,
wherein said comparing provides a determination of said woman's susceptibility to conceive an aneuploid embryo.

12. A system for determining a woman's susceptibility to carry an aneuploid embryo comprising:
a computing environment;
an input device, connected to the computing environment,
to receive data from a user, wherein the data received comprises items of information from a woman to provide a profile for said woman,
comprising information such as the woman's genotype including the presence or absence of a polymorphic allele, the woman's age and, optionally, prospective father's age,
an output device, connected to the computing environment,
to provide information to the user; and
a computer readable storage medium having stored thereon at least one algorithm to provide for comparing the woman's profile to a library of profiles known to provide an indication of a woman's susceptibility to carry an aneuploid embryo,
wherein the system provides results that can be used for determining a woman's susceptibility to carry an aneuploid embryo.
